(19) United States
(12) Patent Application Publication
(10) Pub. No.: US 2022/0372561 A1
(43) Pub. Date: Nov. 24, 2022

(54) METHODS FOR PERFORMING DIGITAL PCR

(71) Applicants: Sepehr Kiani, Watertown, MA (US); Aaron Weber, Watertown, MA (US); Robert Meltzer, Belmont, MA (US)

(72) Inventors: Sepehr Kiani, Watertown, MA (US); Aaron Weber, Watertown, MA (US); Robert Meltzer, Belmont, MA (US)

(73) Assignee: Fluent Biosciences Inc., Watertown, MA (US)

(21) Appl. No.: 17/747,645

(22) Filed: May 18, 2022

Related U.S. Application Data
(60) Provisional application No. 63/190,150, filed on May 18, 2021.

(51) Int. Cl. C12Q 1/6825; C12Q 1/686

(52) U.S. Cl. CPC C12Q 1/6825; C12Q 1/686

(57) ABSTRACT

This invention releases to systems and methods for detecting the presence and quantity of a target nucleic acid in a sample using dPCR and PIP encapsulated monodisperse droplets.

### TECHNICAL FIELD

The present invention relates to bioassays such as digital polymerase chain reaction.

### BACKGROUND OF THE INVENTION

Biological fluids contain a variety of targets useful for diagnostic, research, and medical treatment purposes. Those targets may include liquid biopsy targets such as circulating cells (tumor, fetal, or stem), cellular components (e.g. nuclei), cell-free nucleic acids, extracellular vesicles, and protein antigens. Relevant targets may also include biological targets indicative of disease in a sample, such as prokaryotes, fungi, and viruses. However, the quantitative detection of biological targets, e.g., nucleic acids and proteins, at the single-cell and/or single-molecule level can be challenging due to the need to isolate and assay minute components in a sample.

Recently, an efficient and flexible target-specific approach to capture and label targets of interest from biological samples was described. The approach uses a particle-templated emulsification technique to capture and isolate biomolecules from a sample. Hatori et al., 2018 “Particle-Templated Emulsification for Microfluidics-Free Digital Biology” Anal. Chem., 10.1021/acs.analchem.8b01759. In short, the technique, also known as pre-templated instant partitions (PIPs) encapsulation, uses template particles to capture targets of interest in a sample. The template particles and targets are vortexed in immiscible fluids. As a result, an emulsion of monodispersed droplets that contain a single template particle and target is created.

These monodisperse droplets are an ideal vessel in which to perform isolated reactions, such as nucleic acid amplification reactions, e.g. polymerase chain reactions (PCR). The present Inventors have shown that PIP encapsulation can be used to create an emulsion of monodisperse droplets that each include a single template particle, a target nucleic acid, and reagents necessary for PCR amplification; and that PCR amplification could then take place within the droplets. (WO 2020/069298, incorporated herein by reference). Further, the Inventors showed that these droplets could be made without using complex fluidic handling devices.

In contrast, common, commercially available systems and methods for PCR-based detection assays, such as digital PCR (dPCR), require complex fluidic and signal detecting elements. For example, BEAMing (beads, emulsion, amplification, magnetics), which requires the use of complex fluidics to capture microbeads and perform flow cytometry. The complex fluidics increase costs and limit throughput of these systems. Thus, it becomes difficult to scale up throughput of these systems, especially in multiplex assays using multiple optical reporters.

### SUMMARY OF THE INVENTION

The present invention provides methods and compositions for performing PCR-based detection assays, such as dPCR assays, using an emulsion of monodisperse droplets. The droplets are created using pre-templated instant partition (PIP) encapsulation. By using PIP encapsulation droplets are created that each include a single template particle, a target nucleic acid, and reagents necessary for PCR amplification and detection (e.g., primers and reporters).

Advantageously, these assays can be performed using a simple fluidic protocols and components, obviating the need for complex and expensive microfluidics. Since methods of the invention are unconstrained from the costs and throughput issues caused by complex fluidics, they provide a low cost and scalable modality for conducting multiplex amplification-based detection assays. Moreover, certain methods of the invention can be used to perform a dPCR assay with widely-available, and sometimes repurposed, optical equipment to detect signals from commonly used reporters, such as TaqMan probes.

This simple dPCR assay can not only identify and quantify the presence of a target nucleic acid, but the resulting amplicons used for down-stream applications. For example, target dPCR amplicons can be quickly and easily isolated by selectively recovering droplets emitting a fluorescent signal (e.g., from the dPCR reporter). These amplicons can then, for example, form the basis of a sequencing library, which is then sequenced.

The present invention therefore provides high-throughput, multiplex, and cost-effective systems and methods that identify and quantify one or more target nucleic acids in a sample. Methods include approaches for detecting the presence, and preferably sequence, of a nucleic acid sequence of interest, even at low concentrations. For example, methods of the invention are useful to identify microbial nucleic acids, e.g., those indicative of a bacterial infection, in a sample obtained from a patient.

Moreover, methods of the invention provide approaches to faithfully amplify small amounts of nucleic acids without material loss or significant amplification biases. Methods of the invention use emulsions to isolate, capture, and clonally amplify nucleic acids molecules inside droplets. Droplets are formed using pre-templated instant partitions (PIPs) in which particles template the formation of droplets inside a reaction vessel and segregate individual nucleic acid molecules therein, such that each droplet contains a single template particle and a single nucleic acid molecule. In certain aspects, the template particles capture nucleic acids in a sample using capture oligonucleotides to capture target nucleic acids in a sample prior to droplet formation. However, in preferred aspects of the invention, methods use “blank” or “undecorated” template particles, i.e., they do not include a capture moiety that captures a target nucleic acid prior to droplet formation. Rather, using a ubiquitous, undecorated template particle, individual template particles can be isolated, amplified, and even analyzed in monodisperse droplets.

Each droplet functions as an isolated reaction chamber, thereby ensuring that every nucleic acid molecule has equal access to resources required for amplification. Thus, amplification bias is significantly reduced or eliminated. Moreover, methods of the invention collect and amplify nucleic acid molecules in a single reaction vessel or tube, eliminating the need to transfer the material across multiple vessels or tubes, which prevents or eliminates material loss.

The present invention provides methods to detect a target nucleic acid in a sample. An exemplary method includes obtaining nucleic acid from a biological sample and preparing an aqueous solution comprising target nucleic acid, PCR reagents, template particles, primers specific for the target nucleic acid and fluorescent probes. The aqueous solution is combined with an oil in a vessel to create a mixture, which is sheared to simultaneously form a plurality of water-in-oil partitions, wherein each of the partitions includes a single target nucleic acid, PCR reagents, a template particle, primers specific for the target nucleic acid, and at least one fluorescent probe. Primers and fluorescent probes are hybridized to the target nucleic acid and amplified in the partitions, thereby hydrolyzing the probes to release a fluorescent label. Finally, fluorescent signal is detected to indicate the presence of the target nucleic acid.

In preferred methods, the template particles lack any capture moieties to capture the template particles. In certain aspects, the template particles template the formation of the droplets and segregate the microbial nucleic acid inside one of the droplets away from other nucleic acids present in the sample.

In certain methods, the fluorescent signal is detected using a fluorometer. The fluorometer can be a simple fluorescent cell counter.

Methods of the invention further include quantifying the amount of target nucleic acid in a sample. When quantifying the amount of target nucleic acid, methods of the invention include counting the number of droplets that produce a detectable signal and the number of droplets that do not produce a detectable signal. In preferred methods, the target nucleic acid is loaded into the partitions at a limiting dilution.

In certain aspects, methods of the invention further include detecting the presence of two or more different target nucleic acids in a sample. In such methods, shearing the mixture forms a plurality of partitions that each include one of the different target nucleic acids. In certain aspects, the partitions include a plurality of hydrolysis probes, wherein each probe binds to a different target nucleic acid and includes a different fluorescent label. In certain aspects, identifying the presence of the target nucleic acids in the sample includes imaging the partitions to detect the fluorescence emission of each different fluorescent label. The present invention further provides methods quantifying the amount of each different target nucleic acid in the sample.

In an aspect, the invention provides a method to detect a microbial nucleic acid in a patient sample. The sample may be a blood or plasma sample. Preferably the microbial nucleic acid comprises cell-free DNA, and more preferably, the microbial nucleic acid is cell-free 16S rDNA. The sample may also include other nucleic acids that are not from a microbe, such as, nucleic acids released from the patient's own cells. To isolate the microbial nucleic acid from other nucleic acids, methods of the invention include partitioning the sample to form a plurality of droplets simultaneously in a vessel, wherein the microbial nucleic acid is segregated inside one of the droplets. The microbial nucleic acid is bound with a capture probe inside the droplet, as the capture probe includes a nucleotide sequence that is complementary to the microbial nucleic acid. The bound microbial nucleic acid is subsequently amplified to create an amplicon. The amplicon is detected, thereby detecting the microbial nucleic acid present in the sample.

In preferred embodiments, the microbial nucleic acid is associated with a 16s rDNA gene. The 16s rDNA gene is present in all known microbes and contains a favorable mix of highly conserved regions and hypervariable regions. A genetic element with those characteristics can be used to identify an unknown microorganism by comparing sequence reads to sequences from the same genetic region(s) from known microorganisms (e.g., by aligning to those known sequences and identifying disparities). Accordingly, the microbial nucleic acid is preferably associated with the 16s rDNA gene and can thus be used to detect presence of microbial nucleic acid inside a patient sample and then be sequenced to determine the identity of the microbe.

In preferred embodiments, amplification is performed by PCR in the presence of a fluorophore to create an amplicon with the fluorophore incorporated therein. The fluorophore may include, for example, fluorescently labeled nucleotides or an intercalating dye. During amplification, the fluorophore is incorporated into the amplicon, which allows the resultant amplicon to be easily detected by, for example, measuring for a fluorescent signal from the fluorophore. As such, a sample processed by methods of the invention can be quickly assessed to determine whether the sample contains microbial nucleic acids. For example, the sample may be observed underneath a fluorescent light or device, such as a fluorometer. Amplicons present in the sample emit a fluorescent signal on account of the fluorophores. Because the amplicons are copies of microbial nucleic acids, the fluorescent signal is indicative of microbial nucleic acids present in the sample.

Methods of the invention use droplets to capture and amplify microbial nucleic acids while eliminating interference from non-microbial nucleic acids, thereby preventing amplification biases. This is particularly useful when microbial nucleic acids are present at very small quantities, e.g., as low as about 0.01% frequency of total nucleic acids in a given sample. The droplets may be prepared as emulsions, e.g., as an aqueous phase fluid dispersed in an immiscible phase carrier fluid (e.g., a fluorocarbon oil, silicone oil, ora hydrocarbon oil) or vice versa. Generally, the droplets are formed by shearing two liquid phases. Preferably, the droplets are templated by particles, referred to as template particles. Accordingly, in preferred embodiments, methods of the invention involve combining template particles with the sample in a first fluid adding a second fluid that is immiscible with the first fluid to create a mixture and vortexing the mixture to thereby partitioning the sample and form the plurality of droplets. The template particles template the formation of the droplets and segregate microbial nucleic acid therein.

In preferred embodiments, the template particle comprises the capture probes. The capture probes may be tethered to the template particle and comprises a nucleotide sequence that is complementary to one or more portions of a 16s rDNA gene. The template particle may comprise a plurality of capture probes with nucleotide sequences that are complementary to different portions of 16s rDNA gene, thereby allowing sequences from across a significant portion of the 16s rDNA gene to be captured and profiled.

Template particles according to aspects of the invention may comprise hydrogel, for example, selected from agarose, alginate, a polyethylene glycol (PEG), a polyacrylamide (PAA), acrylate, acrylamide/bisacrylamide copolymer matrix, azide-modified PEG, poly-lysine, polyethyleneimine, and combinations thereof. In certain instances, template particles may be shaped to provide an enhanced affinity a nucleic acid. For example, the template particles may be generally spherical but the shape may contain features such as flat surfaces, craters, grooves, protrusions, and other irregularities in the spherical shape that promote an association with a nucleic such that the shape of the template particle increases the probability of templating a monodisperse droplet that contains a nucleic acid.

Methods of the invention are useful for detecting microbial nucleic acids. The microbial nucleic acids may be any one of RNA, DNA, or both or fragments. The microbial nucleic acids may comprise cell-free nucleic acids, which can be taken from blood or plasma via non-invasive procedures. In preferred embodiments, the microbial nucleic acid is at least one of cell-free 16s rDNA or cell-free 16s rRNA. And more preferably, the microbial nucleic acid is cell-free 16s rDNA, which is more stable than 16s rRNA.

Samples positively identified for microbial nucleic acids are preferably sequenced. Sequencing produces sequence reads that are useful to identify pathogenic microbes. As such, in preferred embodiments, methods include preparing microbial nucleic acids for sequencing. Preparing may involve amplifying the amplicons. The amplicons may be amplified by PCR using primers that incorporate additional primers, such as, P5 and P7 sequencing primers.

Methods of the invention provide approaches for identifying a microbe. The method may involve using a computer system comprising a processor coupled to a memory device for analyzing sequence reads obtained by sequencing microbial nucleic acids as well as sequence information from one or more references. The references may comprise sequence information from different species of microbes and/or different strains of species. Matching the sequence reads to the references can be used to identify the microbe based on similarity. Accordingly, methods may involve aligning the sequence reads to the references and determining an alignment score between the sequence reads and sequences of references of known microbes. Determining the alignment score may include calculating match scores between bases of the sequence reads and bases in the references. An alignment score above a pre-determined threshold is used to reveal a match, and thus identify the microbe. The method may further involve providing a report that includes the identity of the microbe. Based on the identify the microbe, a physician can administer an effective treatment to the patient to kill the microbe and thus alleviate symptoms of sepsis. Other aspects and advantages of the invention are apparent to the skilled artisan upon review of the follow detailed description thereof.

Instruments and Systems

The present invention also provides systems that can include, on a single instrument, all the components necessary to conduct multiplex qPCR and/or dPCR assays, including thermocycle and signal detection components. The systems of the invention can perform multiplex qPCR and dPCR reactions using an emulsion of monodisperse droplets that each include a single template particle, a target nucleic acid, and reagents necessary for PCR amplification. Further, these reactions can be performed using a simple fluidic cartridge, obviating the need for complex microfluidics. Since the systems are unconstrained from the costs and throughput issues caused by complex fluidics, they provide a low cost and scalable modality for conducting multiplex amplification-based detection assays.

The systems and instruments of the invention can include thermal elements as used in thermal cycling applications. In certain aspects, the systems and instruments of the invention include opposing thermal elements that are seated on two sides of a fluidic cartridge using a clamping system. By heating a sample from two sides, especially a sample with a low volume, the systems and instruments of the invention can achieve unprecedented thermal cycle performance for amplification assays.

The system can also include several components used to detect signals from samples in a fluidic cartridge. These components can include, for example, an imaging subsystem (e.g., a camera), illumination zones to illuminate samples and, in certain aspects, excite optical reporters such as fluorescent reporters in a sample. The system can also include a variety of optical elements, such as filters, to allow the system to image a variety of optical labels in a single assay, which permits the systems and instruments of the invention to perform multiplex assays.

In certain aspects, the thermal elements, illumination zones, and optical elements can be disposed on a series of rotating stages. Thus, as the needs of an assay require, the system can rotate the stages to align a sample with thermal elements for thermal cycling. Then, once amplification is complete, the system can rotate the thermal elements away in favor of an illumination zone and optical elements, such that the imaging subsystem can detect signals from a sample. In this way, a single instrument can perform both amplification and signal detection, enabling, for example, the single instrument to perform dPCR assays. Further, the stages can be rotated during imaging to align the sample with different illumination zones and/or optical elements, such as filters. This allows the systems of the invention to image multiple optical reporters in a single assay.

In certain aspects, the invention provides a biological sample handling system. The system includes a first stage and a second stage, each disposed around a central axis. Each stage has a working surface perpendicular to the central axis. The system also includes a fluidic sample cartridge and a cartridge holder for receiving the fluidic cartridge. The cartridge holder can be disposed between the working surfaces of the stages.

The system also includes a first heating element disposed on a perimeter of the working surface of the first stage and a second heating element disposed on a perimeter of the working surface of the second stage. The system can also include clamping system.

In certain aspects, the first and second stages rotate about the central axis to align the first and second heating elements with the fluidic sample cartridge, and the clamping system translates one or more of cartridge holder and stages in a direction parallel to the central axis such that the heating elements contact the fluidic sample cartridge. This seats the thermal elements on the cartridge to permit thermocycling of the sample in the cartridge.

In certain aspects, the system also include a third heating element disposed on the perimeter of the working surface of the first stage and a fourth heating element disposed on the perimeter of the working surface of the second stage. The first and second stages rotate about the central axis to align the third and fourth heating elements with the fluidic sample cartridge, while simultaneously displacing the first and second thermal elements.

In certain aspects, the systems of the invention further include an optical subsystem (e.g., a camera) disposed perpendicular to the first stage and the cartridge holder.

The first stage may include one or more optical element disposed proximal to the perimeter of the working surface; and/or the second stage may include one or more illumination zone disposed proximal to the perimeter of the working surface. Thus, in certain aspects, the first and/or second stage rotates around the central axis to align the cartridge with the optical element, the illumination zone, and the imaging subsystem.

Optical elements can include, for example, one or more of a filter, a lens, a prism, an objective, a mirror, a baffle, a slot, a light dispersal component, and a light blocking components. In certain systems, the first stage comprises a plurality of optical elements disposed around the perimeter of the first stage. Each optical element of the plurality can include a different filter. Each different filter allows a different wavelength of light or range of wavelengths of light to pass through the filter and to the detection subsystem. Thus, the first stage can rotate to align the cartridge and imaging subsystem with a different optical element of the plurality, and thereby detect different wavelengths of light from the sample.

In certain aspects, the illumination zone includes one or more illumination source. An illumination source includes, for example, one or more of an incandescent lamp, a gas discharge lamp, a light emitting diode (LED), an organic LED (OLED), a diode laser bar, a laser, and a diode laser. The second stage may include a plurality of illumination zones disposed along the perimeter of the second stage, and wherein each different illumination zone of the plurality transmits spectrally distinct light. Thus, the second stage can rotate to align the cartridge and imaging subsystem with each different illumination zone.

The systems of the invention also include a fluidic cartridge. The cartridge can include a sample area disposed between a first and second optically transparent substrate, and when the cartridge is received by the cartridge holder the first substrate faces towards the first stage and the second substrate faces towards the second stage. In certain aspects, when the cartridge is aligned with one of the optical elements and one of the illumination zones, the illumination zone transmits excitation through the second substrate to a fluorescent reporter in the sample area causing the fluorescent reporter to transmit emission light through the first substrate to the optical element.

Systems of the invention may also include a control module coupled to a non-transitory, tangible memory and operable to receive an input designating an assay to be performed on a sample in the cartridge and control the instrument to perform the assay. The assay may, for example, be a PCR amplification assay and the control module controls the first and second stages such that one or more thermal elements align with the sample cartridge. In certain aspects, the assay is a digital PCR assay and the control module controls the first and second stages such that an optical element and illumination zone align with the sample cartridge. In certain aspects, the sample includes an emulsion of monodisperse water-in-oil droplets that each comprise a template particle and a nucleic acid template.

### BRIEF DESCRIPTION OF DRAWINGS

FIG. 1 shows a vessel containing nucleic acids and template particles before vortexing.

FIG. 2 shows a vessel containing nucleic acids and template particles inside droplets.

FIG. 3 shows an exemplary method for detecting a target ctDNA in a sample using dPCR in monodisperse droplets.

FIG. 4 shows an image of fluorescently labeled droplets taken with a fluorescent cell counter.

FIG. 5 shows an exemplary capture probe.

FIG. 6 shows a droplet with a target nucleic acid and a template particle.

FIG. 7 shows the droplet with target nucleic acid bound to a capture probe.

FIG. 8 shows an amplicon inside a droplet.

FIG. 9 shows a final sequencing product.

FIG. 10 shows a fluorescent cell counter and an image taken by it during a dPCR assay.

FIG. 11 provides a schematic of an exemplary system of the invention.

FIG. 12 provides a closeup view of a system of the invention.

FIG. 13 provides a closeup view of a system of the invention.

FIG. 14 provides a closeup view of a system of the invention.

FIG. 15A shows a fluid cartridge of the invention FIG. 15B shows a cutaway view of a fluid cartridge of the invention.

FIG. 16 provides a schematic of wide-field wavelength scanning.

FIG. 17 provides a schematic of components of the invention used to perform wide-field wavelength scanning.

FIG. 18 provides a schematic of hyperspectral scanning.

FIG. 19 provides a schematic of components of the invention used to perform hyperspectral scanning.

FIG. 20 provides a schematic of components of the invention used to perform sensor filter scanning.

FIG. 21 shows examples of signal parsing in sensor filter scanning.

FIG. 22 provides a schematic of a sample area in a fluid cartridge as used in certain PCR assays.

### DETAILED DESCRIPTION

The present invention provides methods and compositions for performing PCR-based detection assays, such as dPCR assays, using an emulsion of monodisperse droplets. The droplets are created using pre-templated instant partition (PIP) encapsulation. By using PIP encapsulation droplets can be created that each include a single template particle, a target nucleic acid, and reagents necessary for PCR amplification and detection (e.g., primers and reporters).

Advantageously, in certain methods and systems of the invention, the monodisperse droplets can be formed (including a single template nucleic acid and all necessary PCR reagents) in a single reaction vessel.

The droplets may be prepared as emulsions, e.g., as an aqueous phase fluid dispersed in an immiscible phase carrier fluid (e.g., a fluorocarbon oil, silicone oil, or a hydrocarbon oil) or vice versa. Generally, the droplets are formed by shearing two liquid phases. Shearing may comprise any one of vortexing, shaking, flicking, stirring, pipetting, or any other similar method for mixing solutions.

The droplets all form substantially simultaneously at the moment of shearing the immiscible fluids, generally an aqueous solution and a second fluid, such as an oil. As a result, each droplet provides an aqueous partition, surrounded by oil. In certain aspects, the aqueous solution includes nucleic acids, including a target nucleic acid. The aqueous solution can also include all the reagents necessary for carrying out a PCR-based detection assay, e.g., PCR amplification reagents (e.g., dNTPs, primers, and polymerase) and detectable reporters, such as fluorescent probes. This entire solution can be combined with an oil and vortexed to simultaneously create an emulsion with monodisperse droplets that contain a single target nucleic acid template. Also or alternatively reagents, such as, DNA polymerase, may be delivered directly into droplets via the template particles to ensure each droplet receives a substantially uniform quantity of reagents. Once the droplets are prepared PCR amplification and target nucleic acid detection can occur in the droplets.

A feature of certain methods as described herein is the use of a polymerase chain reaction (PCR)-based assay to detect the presence of certain oligonucleotides and/or genes of interest in a sample. Exemplary target nucleic acids include those associated with genetic mutations or diseases in a subject. Other target nucleic acids include, for example, those associated with a viral or bacterial infection.

The systems and methods of the invention include using PIP encapsulated droplets to perform a number of partitioned PCR-based detection assays, which include quantitative PCR (qPCR), quantitative fluorescent PCR (QF-PCR), multiplex fluorescent PCR (MF-PCR), digital PCR (dPCR), PCR-RFLP/real time-PCR-RFLP, hot start PCR, nested PCR, in situ polony PCR, in situ rolling circle amplification (RCA), bridge PCR, picotiter PCR, and reverse transcriptase PCR (RT-PCR).

In such assays, one or more primers specific to each target nucleic acid or gene of interest are reacted with the genome of each cell. These primers have sequences specific to the particular target, so that they will only hybridize and initiate PCR when they are complementary to the target. If the target of interest is present and the primer is a match, many copies of the target are created using PCR amplification. To determine whether a particular target is present in a droplet, the PCR products may be detected through an assay probing the liquid of the monodisperse droplet, such as by staining the solution with an intercalating dye, like SybrGreen or ethidium bromide, hybridizing the PCR products to a solid substrate, such as a bead (e.g., magnetic or fluorescent beads, such as Luminex beads), or detecting them through an intermolecular reaction, such as FRET using fluorescent probes or using fluorescent hydrolysis probes. These dyes, beads, probes and the like are each used to detect the presence or absence of nucleic acid amplification products, e.g., PCR products.

PCR- and real-time PCR-based detection methodologies have greatly improved the analysis of nucleic acids from both throughput and quantitative perspectives. Traditional PCR-based detection assays generally rely on end-point, and sometimes semi-quantitative, analysis of amplified DNA targets via agarose gel electrophoresis, real-time PCR (or qPCR) methods are most often used to quantify exponential amplification as the reaction progresses. Quantitative PCR reactions are monitored either using a variety of highly sequence specific fluorescent probe technologies, or by using non-specific DNA intercalating fluorogenic dyes.

Preferred systems and methods of the invention include dPCR. Digital PCR (dPCR) is an alternative quantitation method in which target nucleic acids from a dilute sample are individually isolated droplets using PIP encapsulation. The isolated target nucleic acids are amplified in separate reactions in each droplet. The distribution from background of target DNA molecules among the reactions follows Poisson statistics at the terminal and/or limiting dilutions of target DNA. Generally, at a terminal dilution the vast majority of droplets contain either one or zero target DNA molecules. Ideally, at terminal dilution, the number of PCR positive reactions (PCR(+)) equals the number of template molecules originally present. At a limiting dilution, partitions include zero, one, and often more than one target nucleic acid following the Poisson distribution. At the limiting dilution, Poisson statistics are used to uncover the underlying amount of target DNA originally present in a sample.

Thus, methods and systems of the invention involve forming PIP encapsulated droplets where some droplets contain zero target nucleic acid molecules, some droplets contain one target nucleic acid molecule, and some droplets may or may not contain multiple nucleic acid molecules (e.g., using limiting or terminal dilutions). In preferred methods and systems, the distribution of molecules within PIP encapsulated droplets obeys the Poisson distribution. However, methods using non-Poisson loading of droplets are contemplated within the scope of the invention and may include, for example, active sorting of droplets, such as by laser-induced fluorescence.

When using a limiting dilution to quantify target nucleic acids, it is preferred that the target nucleic acid sample is diluted to a terminal dilution, such that the vast majority of PIP encapsulated droplets include only a single target nucleic acid or no target nucleic acid (and not multiple target nucleic acids). In certain instances, where a target nucleic acid is present in a sample at a high concentration and/or frequency the emulsion must contain a large number of droplets to accommodate loading all the target nucleic acids at a terminal dilution. Advantageously, the systems and methods of the invention can be quickly scaled to accommodate and analyze large number of monodisperse droplets (e.g., at least 100, at least 1,000, at least 1,000,000, at least 10,000,000 or more droplets).

To perform dPCR, the droplet-isolated target nucleic acids may be detected using labeled probes, such as hydrolysis probes. Exemplary hydrolysis probes include, for example, TaqMan probes produced by Thermo Fisher Scientific. TaqMan probes include an oligonucleotide that binds to a specific sequence in the target nucleic acid. The probes include a detectable label, such a fluorescent dye, and a quencher. When attached to the probe, any signal produced by the fluorescent dye is quenched due the proximity of the dye to the quencher. During PCR amplification, exonuclease activity by a polymerase hydrolyzes the probe hybridized to the target nucleic acid. This, in turn, releases the fluorescent dye from the quencher, allowing it to produce a detectable signal indicative of a polymerase (amplification) reaction. As amplification progresses, probes in the droplets can bind to the resulting amplicons. If these are likewise amplified, the probes hydrolyze and increase the resulting fluorescent signal.

During imaging, partitions that produce a fluorescent signal from the released dyes are marked as a “1” or “0” (positive or negative for amplification), which informs the name “digital” PCR. Absolute quantification of the starting target nucleic acid in a sample can be calculated based on the ratio of PCR positive or negative partitions using Poisson statistics.

A principle advantage of digital compared to qPCR is that it avoids any need to interpret analog signals, i.e., real-time fluorescence versus temperature curves. Moreover, qPCR generally requires a standard curve, preferably from an on-chip standardization reaction to provide quantitative results. Digital PCR forgoes these complications, while still providing an absolute quantification.

In certain aspects, methods and systems of the invention are used to perform diagnostic assays to quantify and/or detect the presence of a nucleic acid associated with a disease or other pathology. In certain aspects, the target nucleic acid is from a cell (e.g., circulating cells and/or circulating tumor cells), a virus, bacteria, or one or more genes of interest or genetic markers (e.g., oncogenes, or heterogeneous genes in a sample).

Generally, methods of the invention include the steps of obtaining a sample containing a target nucleic acid and preparing an aqueous solution comprising the target nucleic acid, PIP template particles, and the reagents necessary for a PCR-based detection assay, e.g., PCR amplification reagents and fluorescent probes. Then, the aqueous solution is contacted with a second, immiscible fluid, such as an oil.

PIP encapsulation involves preparing an emulsion, e.g., an aqueous phase fluid dispersed in an immiscible phase carrier fluid (e.g., a fluorocarbon oil, silicone oil, or a hydrocarbon oil) or vice versa. Generally, the droplets are formed by shearing two liquid phases. The template particles template formation of the droplets during shearing. Accordingly, in preferred embodiments, methods of the invention involve combining template particles with the sample in a first fluid adding a second fluid that is immiscible with the first fluid to create a mixture and vortexing the mixture to thereby partition the sample into a plurality of droplets. The template particles template the formation of the droplets and segregate a target nucleic, PCR reagents, and detectable reporters into individual droplets.

Preferably, methods of the invention include vortexing the vessel containing the sample the aqueous solution and immiscible fluid. Vortexing is preferably done by pressing the vessel onto a vortexer, which creates sufficient shear forces inside the vessel to partition the aqueous fluid into monodisperse droplets. After vortexing, a plurality monodisperse droplets (e.g., at least 100, at least 1,000, at least 1,000,000, at least 10,000,000 or more) are formed essentially simultaneously.

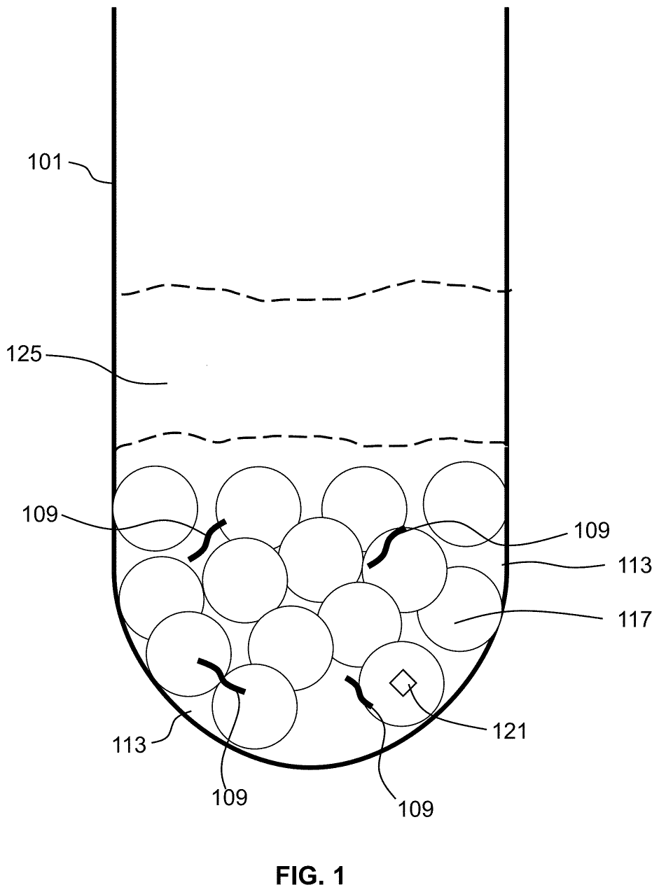

FIG. 1 shows a vessel 101 containing a target nucleic acid 109 and template particles 117 before vortexing. The vessel 101 includes a mixture of nucleic acids and template particles 117 inside an aqueous fluid 113 with an oil overlay. The aqueous fluid 113 may include reagents, such as, reagents for preserving samples of nucleic acids, e.g., EDTA, or for nucleic acid synthesis, such as, reagents for PCR. In some embodiments, one or more of reagents may be provided by template particles 117. Accordingly, template particles 117 may include one or more compartments 121 containing the reagents, which are releasable from the compartments 121 in response to an external stimulus, such as, for example, heat, osmotic pressure, or an enzyme. Reagents may include nucleic acid synthesis reagents, such as, for example, a polymerase, primers, dNTPs, fluorophores, or buffers. In addition, the vessel 101 further includes a second fluid 125 that is immiscible with the first fluid, e.g., an oil.

In some aspects, generating the template particles-based monodisperse droplets involves shearing two liquid phases. The liquid phase comprising template particles and target nucleic acids is the aqueous phase and, in some embodiments, the aqueous phase may further include reagents selected from, for example, buffers, salts, lytic enzymes (e.g. proteinase k) and/or other lytic reagents (e. g. Triton X-100, Tween-20, IGEPAL, bm 135, or combinations thereof), nucleic acid synthesis reagents e.g. nucleic acid amplification reagents. The second phase is a continuous phase and may be an immiscible oil such as fluorocarbon oil, a silicone oil, or a hydrocarbon oil, or a combination thereof. In some embodiments, the fluid may comprise reagents such as surfactants (e.g. octylphenol ethoxylate and/or octylphenoxypolyethoxyethanol), reducing agents (e.g. DTT, beta mercaptoethanol, or combinations thereof). For example, see Hatori et. al., Anal. Chem., 2018 (90):9813-9820, which is incorporated by reference.

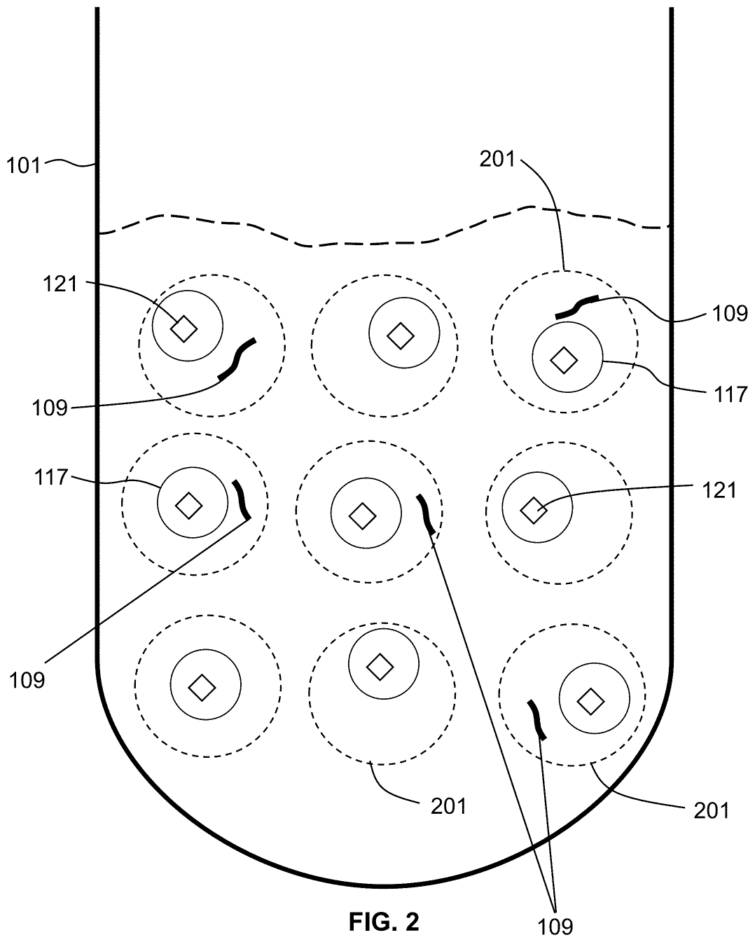

FIG. 2 shows a vessel 101 containing target nucleic acids 109 and template particles 117 inside droplets. The vessel 101 includes a plurality of monodisperse droplets 201, at least one of which contains a single fragment of the target nucleic acid, 109, and a temple particle 117. A person of skill in the art will recognize that not all of the droplets 201 generated according to aspects of the invention will necessarily include a single one nucleic acid and a single one of the template particles. In some instances, a droplet 201 may include more than one, or none, the nucleic acids or template particles. Droplets that do not contain one of each nucleic acid and a template particle may be removed from the vessel 101, destroyed, or otherwise ignored. In some instances, template particles 117 may be formulated so as to have a positive surface charge, or an increased positive surface charge. Such materials may be without limitation poly-lysine or polyethyleneimine, or combinations thereof. This increases the probability of an association between the template particle 117 and the target nucleic acid, which is negatively charged.

In certain aspects, the aqueous solution includes reagents necessary for a PCR-based detection assay, such as dPCR. Such reagents generally include Taq polymerase, deoxynucleotides of type A, C, G and T, magnesium chloride, and forward and reverse primers, detectably labeled probes (e.g., hydrolysis probes) all suspended within an aqueous buffer.

In certain aspects, the isolated amplification reactions in the PIP encapsulated droplets are detected using one or more detectable probes and/or primers. Preferably, detectable probes and/or primers are optically detectable, for example, using fluorescent labels.

Examples of fluorescent labels include, but are not limited to, Atto dyes, 4-acetamido-4′-isothiocyanatostilb ene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′, 6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-(DTAF), 2′, 7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamineB sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAIVIRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalocyanine; and naphthalo cyanine. Labels other than fluorescent labels are contemplated by the invention, including other optically-detectable labels.

In order to detect and quantify multiple target nucleic acids in a sample, one or more distinct detectable labels can be used, e.g., a different label on different probes. In preferred methods, when multiple different fluorescent reporters are used, the reporters include a combination of one or more of Fam, Yamima Yellow (YAK), SUN, HEX, Cy3, Cy5, TEX, TYE, ATTO dyes, and Alexa dyes. In certain methods, three dyes are used and include Fam, Cy3, and Cy5. Preferably, the fluorescent dyes can be quenched using a fluorescent quencher. In preferred aspects, the different fluorescent labels have emission spectrum that can be readily distinguished.

In certain aspects, the droplets contain a plurality of detectable probes that hybridize to amplicons/target nucleic acids produced in the droplets. Members of the plurality of probes can each include the same detectable label, or a different detectable label (e.g., in the case of multiplex assays to detect multiple target nucleic acids). The plurality of probes can also include one or more groups of probes at varying concentration. The groups of probes at varying concentrations can include the same detectable label which vary in intensity, due to varying probe concentrations. In such methods, a single fluorescent label can be used with different probe sequences to detect/quantify multiple target sequences in a sample.

Methods and systems of the invention are not limited to the TaqMan assay, as described above, but rather the invention encompasses the use of all fluorogenic DNA hybridization probes, such as molecular beacons, Solaris probes, scorpion probes, and any other probes that function by sequence specific recognition of target DNA by hybridization and result in increased fluorescence on amplification of the target sequence.

In certain aspects, amplicons are detected in droplets using an intercalating dye, such as, SYBR Green. During amplification, the fluorophore is incorporated into an amplicon, which allows the resultant amplicon to be easily detected by measuring for a fluorescent signal from the fluorophore. As such, a sample processed by methods of the invention can be quickly assessed to determine whether the sample contains copies of target nucleic acids.

In certain aspects, fluorescent signals from droplets of a sample are observed underneath a fluorescent light or device, such as, a fluorometer. A fluorometer or fluorimeter is a device used to measure parameters of visible spectrum fluorescence: its intensity and wavelength distribution of emission spectrum after excitation by a certain spectrum of light. These parameters are used to identify the presence and the amounts of specific molecules in a medium. Modern fluorometers are capable of detecting fluorescent molecule concentrations as low as 1 part per trillion. In certain aspects, the fluorometer is a fluorescent cell counter.

In some embodiments, the droplets are lysed to release the fluorescently labeled amplicons prior to detection. After lysing the droplets, the sample may undergo one or more washing steps to rid the sample of fluorophores not incorporated inside DNA and thus make it easier to detect the presence of amplicons. Preferably, in such methods, the amplicons are attached to the template particle, e.g., using a capture oligonucleotide. Any amplicons present in the sample will emit a fluorescent signal on account of the fluorophores.

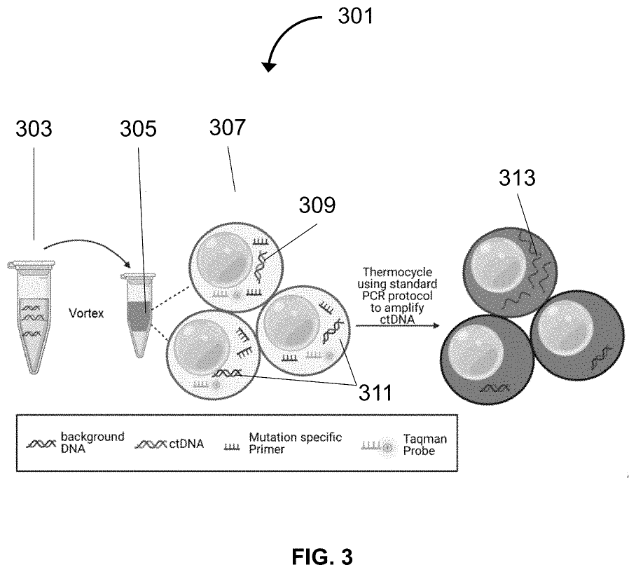

FIG. 3 provides an exemplary method 301 of the invention using dPCR to detect circulating tumor DNA (ctDNA) from a sample. The presently disclosed methods are readily amenable to amplifying and detecting a target nucleic acid, even when present at small quantities in a sample. Methods of the invention can be used to amplify and detect target nucleic acids, even when they makeup as low as at a 0.01% frequency amongst all nucleic acid fragments in a sample. Thus, the presently disclosed methods can be used to amplify and detect nucleic acids such as cell free DNA (cfDNA) and ctDNA, which are often present at only small concentrations in a sample.

In the exemplary method 301, an aqueous solution 303 is prepared. The solution contains DNA from a sample, including target ctDNA, undecorated PIP template particles, a target specific (mutation specific) primer, and a hydrolysis probe, such as a TaqMan probe. The aqueous solution 303 is combined with an immiscible fluid, such as an oil, and vortexed. This shears the liquid and leads to the production of an emulsion 305 of PIP encapsulated monodisperse droplets 307. Preferably, the ctDNA is diluted to a terminal dilution, such that the vast majority of droplets 307 contain a single target ctDNA 309 or no target ctDNA. Droplets without the target may include background DNA 311.

The hydrolysis probe includes an oligonucleotide that binds to a specific sequence on a target ctDNA molecule. The probe also includes a detectable label, such a fluorescent dye, and a quencher. When attached to the probe, any signal produced by the fluorescent dye is quenched due the proximity of the dye to the quencher. During PCR amplification, exonuclease activity by a polymerase hydrolyzes the probe hybridized to the target nucleic acid. This, in turn, releases the fluorescent dye from the quencher, allowing it to produce a detectable signal indicative of a polymerase (amplification) reaction.

The mutation/target specific primer selectively hybridizes to a target sequence in the ctDNA. When the droplets 307 are contacted with the appropriate conditions, e.g., thermal cycling, the primers are extended to produce copies of the target nucleic acid. Only droplets 307 with the target nucleic acid 309 undergo amplification. As the activity of the PCR polymerase is required to hydrolyze the probe to release the fluorescent reporter, only droplets 307 with the target nucleic acid 309 produce a detectable signal 313.

The droplets with a fluorescent emission from the reporters can be counted, for example, using an automated fluorescent cell counter.

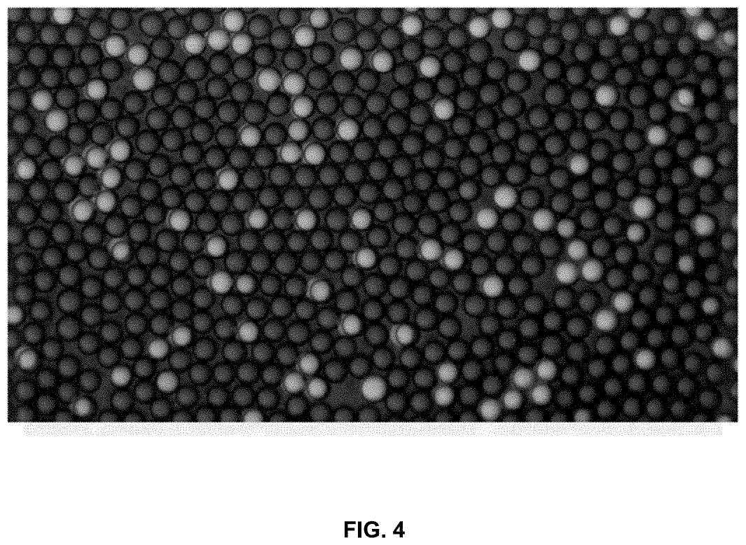

FIG. 4 provides an image of dPCR PIP encapsulated droplets taken using a fluorescent cell counter. The lighter droplets are those emitting a signal from a fluorescent reporter. Thus, the presently disclosed systems and methods can be used with relatively simple optical equipment.

Methods and systems of the invention can be used to detect and quantify target nucleic acids obtained from a variety of sources. For example, target nucleic acids can be obtained from a solid tissue sample or a fluid sample, such as, blood or plasma. Preferably the sample is a fluid sample. Suitable samples may include whole or parts of blood, plasma, cerebrospinal fluid, saliva, tissue aspirate, microbial culture, uncultured microorganisms, swabs, or any other suitable sample. For example, in some embodiments, a blood sample is obtained (e.g., by phlebotomy) in a clinical setting. Whole blood may be used, or the blood may be spun down to isolate the target nucleic acids.

Preferably, the sample is a blood sample. Obtaining the sample may include performing a blood draw to obtain blood or receiving blood from a clinical facility. In some embodiments, obtaining a sample involves a phlebotomy procedure and collects blood into blood collection tube such as the blood collection tube sold under the trademark VACUTAINER by BD (Franklin Lakes, N.J.) or a cell-free DNA blood collection tube such as that sold under the trademark CELL-FREE DNA BCT by Streck, Inc. (La Vista, Nebr.). Any suitable collection technique or volume may be employed. A 10 ml sample of blood from a patient infected with a pathogenic microbe may contain only about 1 ng of microbial nucleic acids.

A target nucleic acid may be RNA, DNA, or a mixture thereof. In certain aspects, the methods of the invention include performing reverse transcriptase reaction to produce cDNA of a target RNA. The reverse transcriptase reaction can be performed in the monodisperse droplets. The resulting cDNA can be amplified and detected as described herein. In preferred aspects, the target nucleic acid is a cell-free nucleic acid, which is preferable because it may be taken from blood or plasma via non-invasive procedures.

In certain aspects, methods of the invention include identifying the presence of one or more target nucleic acids in a sample using dPCR, and sequencing the resulting amplicons. A dPCR reaction can be used to determine whether a sample is positive for the target nucleic acids. Samples negative for target nucleic acids do not need to be sequenced. As such, methods of the invention are useful for identifying samples with target nucleic acids for sequencing analysis. This reduces the amount of sequencing performed, thereby reducing sequencing costs.

Methods may include attaching adaptors to amplicons and/or barcoding target fragments to prepare for downstream sequencing analysis. Any suitable methods may be used to barcode target fragments inside droplets for sequencing. Suitable approaches to attached barcodes to target fragments may include (i) fragmentation and adaptor-ligation (in which adaptors include barcodes); (ii) tagmentation (using transposase enzymes or transpososomes including those sold in kits such as those tagmentation reagent kits sold under the trademark NEXTERA by Illumina, Inc.); and (iii) amplification by, e.g., polymerase chain reaction (PCR) using primers with a hybridization portion complementary to a known or suspected target of interest in a genome and at least one barcode portion that is copied into the amplicons by the PCR reaction. For any of these approaches, the barcodes (e.g., within amplification primers or ligatable adaptors) may be provided free an in solution or bound to a template particle as described herein. In some embodiments, the barcodes are provided as a set (e.g., including thousands of copies of a barcode) in which each barcode is covalently bound to a template particle.

As used herein, barcode generally refers to an oligonucleotide that includes an identifier sequence that can be used to identify sequence reads originating from target nucleic acids that were barcoded as a set with copies of one barcode unique to that set. Barcodes generally include a known number of nucleotides in the identifier sequence between about 2 and about several dozen or more. The oligonucleotides that include the barcodes may include any other of a number of useful sequences including primer segments (e.g., designed to hybridize to a target of interest in a genetic material), universal primer binding sites, restriction sites, sequencing adaptors, sequencing instrument index sequences, others, or combinations thereof. For example, in some embodiments, barcodes of the disclosure are provided within sequencing adaptors such as within a set of adaptors designed for use with a next generation sequencing (NGS) instrument such as the NGS instrument sold under the trademark HISEQ by Illumina, Inc. Within an NGS adaptor, the barcode may be adjacent to the index portion or the target sequence such that the barcode sequence is found in the index read or the sequence read.

In some aspects, a template particle may include capture probes with portions that hybridize or ligate to a target nucleic acid. The capture probe may include any fragment (usually 50-250 bases long) of DNA or RNA which can bind a complementary target nucleic acid, via Watson-Crick base pairing, and also bind with at least one other material (e.g., antibody, a bead, a particle, etc.). Preferably, the capture probe is bound with one of the template particles. The capture probe includes sequences complementary to the target nucleic acid. Thus, the capture probe can bind the target nucleic acid to the template particle. Preferably, the capture probes include a barcode that can, for example, identify the target nucleic acid and/or the droplet/particle to which it was attached.

Generally, the capture probes are oligonucleotides. The capture probes may be attached to the template particle's material, e.g. hydrogel material, via covalent acrylic linkages. In some embodiments, the capture probes are acrydite-modified on their 5′ end (linker region). Generally, acrydite-modified oligonucleotides can be incorporated, stoichiometrically, into hydrogels such as polyacrylamide, using standard free radical polymerization chemistry, where the double bond in the acrydite group reacts with other activated double bond containing compounds such as acrylamide. Specifically, copolymerization of the acrydite-modified capture probes with acrylamide including a crosslinker, e.g. N,N′-methylenebis, will result in a crosslinked gel material comprising covalently attached capture probes. In some other embodiments, the capture probes comprise acrylate terminated hydrocarbon linker and combining the said capture probes with a template particle will cause their attachment to the template particle.

The capture probe may comprise one or more of a primer sequence, a barcode unique to each droplet, a unique molecule identifier (UMI), and a capture sequence.

Primer sequences may comprise a binding site, for example a primer sequence that would be expected to hybridize to a complementary sequence, if present, on any target nucleic acid molecule and provide an initiation site for a reaction, for example an elongation or polymerization reaction. The primer sequence may also be a “universal” primer sequence, i.e. a sequence that is complementary to nucleotide sequences that are very common for a particular set of nucleic acid fragments. The primer sequences used may be P5 and P7 primers as provided by Illumin, Inc., San Diego, Calif. The primer sequence may also allow the capture probe to bind to a solid support, such as a template particle.

By providing capture probes comprising the barcode unique to each droplet, the capture probes may be used to tag the nucleic molecules inside droplets with the barcode, which can, for example, identify the template particle and/or target nucleic acid.

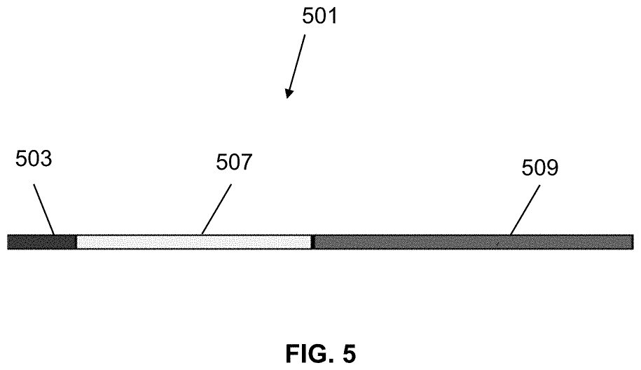

FIG. 5 shows an exemplary capture probe 501. Preferably, the capture probe 501 is attached to a template particle (not shown). The capture probe may include any number of primer binding sites and one or more barcodes. Preferably, the capture probe 501 includes a universal primer for sequencing. For example, the capture probe may include a P5 (503) or P7 primer sequence. The capture probe may further include one or more barcodes 507. The barcode may be a UMI. The capture probe 501 further includes a sequence complementary to a target nucleic acid 509.

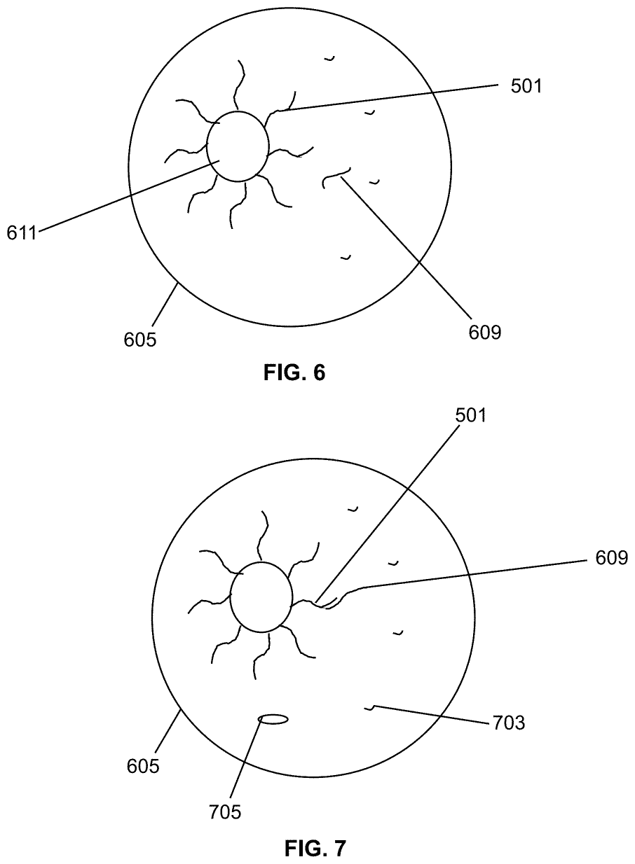

FIGS. 6-8 illustrate a method of preparing target nucleic acids for sequencing using capture probes.

FIG. 6 shows a droplet 605 with a target nucleic acid 609 and a template particle 611. The droplet 605 is formed by making an emulsion with template particles 611 and nucleic acids, including the target nucleic acid 609, inside a vessel. Shown, is a single representative droplet 605 with a template particle 611 and target nucleic acid 609; although, the vessel could contain hundreds to millions of droplets. The template particle 611 includes a plurality of capture probes 501, for example, as described in FIG. 5. The capture probes 501 are tethered to the template particle 611. The capture probes include sequences complementary to the target nucleic acid 609. Accordingly, under conditions that favor hybridization, the target nucleic acid 609 binds to the capture probe 501 via complementary base pairing.

FIG. 7 shows the droplet 605 with target nucleic acid bound to a capture probe. Shown, is the droplet of FIG. 6, at a second time point, after the microbial nucleic acid 609 has hybridized to the capture probe 501. After hybridization, the microbial nucleic acid is amplified by, for example, PCR. Preferably, amplification is performed in the presence of a fluorophore 705. A fluorophore is a fluorescent chemical compound that can re-emit light upon light excitation. The fluorescent dye may be a part of a probe, such as a hydrolysis probe. In certain aspects, the fluorophore 705 is incorporated into an amplicon (e.g., as an intercalating dye), which is made by copying the bound microbial nucleic with a polymerase, e.g., a DNA polymerase. The presence of the fluorophore allows the amplicon to be detected. Primers 703 for PCR may be included in the mixture. The primers 703 may comprise random sequences for binding to the bound target nucleic acid.

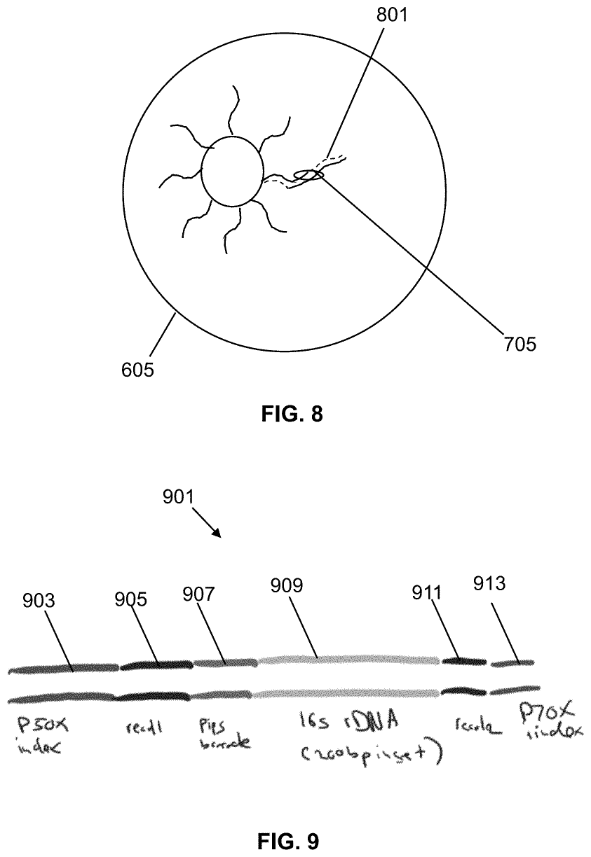

FIG. 8 shows an amplicon 801 inside a droplet 605. In certain aspects, the amplicon 801 includes the fluorophore 705 incorporated therein. The droplet may be lysed to release the template particles bound with capture probes comprising the amplicons 801. A fluorometer may then be used to detect amplicons, and as such, detect the microbial nucleic acids present in the sample based on fluorescence. In some embodiments, the final sequencing product is created by amplifying the amplicon 801 with PCR. PCR may be performed with primers for incorporating one or more additional sequencing primers into the final sequencing product, e.g., as tailed adapters.

FIG. 9 shows a final sequencing product 901. The sequencing product includes, from left to right, a P50X index 903, a Read 1 sequence 905, a barcode 907, the microbial nucleic acid 909, a Read 2 sequence 911, and a P70X index sequence 913.

In certain aspects, the capture probes may include a binding site sequence P5, and an index. The capture probes may further include a binding sequence P7 and a hexamer. Any suitable sequence may be used for the P5 and P7 binding sequences. For example, either or both of those may be arbitrary universal priming sequences (universal meaning that the sequence information is not specific to the naturally occurring genomic sequence being studied, but is instead suited to being amplified using a pair of cognate universal primers, by design). The index segment may be any suitable barcode or index such as may be useful in downstream information processing. It is contemplated that the P5 sequences, the P7 sequence, and the index segment may be the sequences use in NGS indexed sequences such as performed on an NGS instrument sold under the trademark ILLUMINA, and as described in Bowman, 2013, Multiplexed Illumina sequencing libraries from picogram quantities of DNA, BMC Genomics 14:466, incorporated by reference. The hexamer segments may be random hexamers or selective hexamers (aka not-so-random hexamers). Preferably, the template particles are linked to the capture oligos that include one or more primer binding sequences. However, in other aspects, the capture oligos may be released from the template particles prior to attachment with the target fragment.

The template particles of the present disclosure may be prepared using any method known in the art. Generally, the template particles are prepared by combining hydrogel material, e.g., agarose, alginate, a polyethylene glycol (PEG), a polyacrylamide (PAA), Acrylate, Acrylamide/bisacrylamide copolymer matrix, and combinations thereof. Following the formation of the template particles they are sized to the desired diameter. In some embodiments, sizing of the template particles is done by microfluidic co-flow into an immiscible oil phase.

In some embodiments of the template particles, a variation in diameter or largest dimension of the template particles such that at least 50% or more, e.g., 60% or more, 70% or more, 80% or more, 90% or more, 95% or more, or 99% or more of the template particles vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01.

Template particles may be porous or nonporous. In any suitable embodiment herein, template particles may include microcompartments (also referred to herein as “internal compartment”), which may contain additional components and/or reagents, e.g., additional components and/or reagents that may be releasable into monodisperse droplets as described herein. Template particles may include a polymer, e.g., a hydrogel. Template particles generally range from about 0.1 to about 1000 μm in diameter or larger dimension. In some embodiments, template particles have a diameter or largest dimension of about 1.0 μm to 1000 μm, inclusive, such as 1.0 μm to 750 μm, 1.0 μm to 500 μm, 1.0 μm to 250 μm, 1.0 μm to 200 μm, 1.0 μm to 150 μm 1.0 μm to 100 μm, 1.0 μm to 10 μm, or 1.0 μm to 5 μm, inclusive. In some embodiments, template particles have a diameter or largest dimension of about 10 μm to about 200 μm, e.g., about 10 μm to about 150 μm, about 10 μm to about 125 μm, or about 10 μm to about 100 μm.

In practicing the methods as described herein, the composition and nature of the template particles may vary. For instance, in certain aspects, the template particles may be microgel particles that are micron-scale spheres of gel matrix. In some embodiments, the microgels are composed of a hydrophilic polymer that is soluble in water, including alginate or agarose. In other embodiments, the microgels are composed of a lipophilic microgel.

In other aspects, the template particles may be a hydrogel. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), acrylamide/bisacrylamide copolymer matrix, polyacrylamide/poly(acrylic acid) (PAA), hydroxyethyl methacrylate (HEMA), poly N-isopropylacrylamide (NIPAM), and polyanhydrides, poly(propylene fumarate) (PPF).

In some aspects, the presently disclosed template particles further comprise materials which provide the template particles with a positive surface charge, or an increased positive surface charge. Such materials may be without limitation poly-lysine or Polyethyleneimine, or combinations thereof. This may increase the chances of association between the template particle and, for example, and a target nucleic acid.

Other strategies may be used to increase the chances of templet particle-target nucleic association, which include creation of specific template particle geometry. For example, in some embodiments, the template particles may have a general spherical shape, but the shape may contain features such as flat surfaces, craters, grooves, protrusions, and other irregularities in the spherical shape.

Any one of the above described strategies and methods, or combinations thereof may be used in the practice of the presently disclosed template particles and method for targeted library preparation thereof. Methods for generation of template particles, and template particles-based encapsulations, were described in International Patent Publication WO 2019/139650, which is incorporated herein by reference.

In preferred systems and methods, the template particles are undecorated, i.e., they do not contain any moiety that captures a target nucleic acid from a sample. Rather, merely preparing PIP encapsulated droplets as described above causes the target nucleic acids to isolate within the droplets.

In additional methods and systems of the invention, one or more capture oligonucleotides are attached to the template particles. The capture oligonucleotides can be used, for example, to capture the target nucleic acid sequence and/or resulting amplicons. In certain aspects, the capture oligonucleotides only capture amplicons and/or the target sequence after formation of the droplet.

In certain aspects, the target nucleic acid can be a microbial nucleic used to detect the presence of a microbe in a sample. In preferred embodiments, the microbial nucleic acid includes at least one of cell-free 16s rDNA or cell-free 16s rRNA. And more preferably, the microbial nucleic acid is cell-free 16s rDNA, which is more stable than 16s rRNA.

Pathogenic microbes (i.e., microorganisms) infect patients. The body's response to the infection can cause sepsis, which may be life-threatening. Effective treatment may require knowing the identity of the microbe, e.g., the species of microbe. High throughput sequencing represents a powerful approach for identifying pathogenic microbes. For example, microbes can be identified by sequencing nucleic acids isolated from patient blood samples to reveal nucleotide sequences that correspond with specific microbial species. But this approach remains too costly to be applied to too many samples in multiplexed sequencing reactions and the bioinformatic treatment is still not trivial. Moreover, because microbial nucleic acids are present at low concentrations, high-throughput sequencing analyses are often unreliable because of amplification biases that cause over amplification of some non-microbial nucleic acids, leaving the microbial nucleic acids to go undetected. Methods of the invention overcome these limitations by using capture probes that specifically capture and amplify microbial nucleic acids inside droplets.

In particular, certain methods of the invention involve probing nucleic acid samples from patients using probes with nucleotide sequences that are specific to microbial nucleic acids. The nucleotide sequences of the probes are highly specific in binding to complementary microbial nucleic acids and are thus useful to determine whether microbial nucleic acid is present in a patient sample.

Any complementary microbial nucleic acids present in the sample and isolated in PIP encapsulated monodisperse droplets bind to the probes for detection and/or capture. The microbial nucleic acids act as a template for PCR amplification inside the monodisperse droplets. In certain methods, the resulting amplicons can be readily detected and/or isolated. Because the amplicons are copies of the microbial nucleic acids, detection of the amplicons reveals the presence of microbial nucleic acids inside the patient sample.

In certain aspects, samples positive for microbial nucleic acids may be sequenced to identify the species of microbe. A dPCR reaction can be used to determine whether a sample is positive for the microbial nucleic acids. Samples negative for microbial nucleic acids do not need to be sequenced. As such, methods of the invention are useful for identifying samples with microbial nucleic acids for sequencing analysis. This reduces the amount of sequencing performed, thereby reducing sequencing costs.

The microbial nucleic acid can be any nucleic acid useful for detecting a microbe. The nucleic acid may be RNA, DNA, or a mixture thereof. Preferably, the microbial nucleic acid comprises a cell-free nucleic acid, which is preferable because it may be taken from blood or plasma via non-invasive procedures. In preferred embodiments, the microbial nucleic acid includes at least one of cell-free 16s rDNA or cell-free 16s rRNA. And more preferably, the microbial nucleic acid is cell-free 16s rDNA, which is more stable than 16s rRNA.

In certain aspects, the microbial nucleic acid is associated with the 16s rDNA gene. The 16S rDNA gene (or 16S ribosomal DNA gene) is a component of the 30S small subunit of a prokaryotic ribosome that binds to the Shine-Dalgarno sequence. The genes coding for it are referred to as 16S rDNA gene and are used in reconstructing phylogenies, due to the slow rates of evolution of this region of the gene. The 16srDNA gene is present in all known microbes and contains a favorable mix of highly conserved regions and hypervariable regions. A gene with those characteristics can be used to identify an unknown organism by comparing the sequence to sequences from the same gene from known organisms (e.g., by aligning to those known sequences and identifying disparities). Accordingly, nucleic acids associated with the 16s rDNA gene, e.g., 16s rDNA, can be used to detect presence of microbial nucleic acids inside a sample and then sequenced to determine the identity of the microbe.

In most instances, samples from which microbial nucleic acids are obtained will include nucleic acids released from the patient's own cells. These nucleic acids are not helpful for identifying the microbe and may in fact interfere with detection by obscuring the presence of microbial nucleic acids in the sample. As such, it is preferable to isolate the microbial nucleic acid away from other nucleic acids present in the sample. To isolate the microbial nucleic acid from other nucleic acids, methods of the invention include partitioning the sample to form a plurality of droplets simultaneously in a vessel, wherein the microbial nucleic acid is segregated inside one of the droplets.

In certain aspects, the capture probes are attached to template particles. The capture probes may be tethered to the template particles at a 5′ end of the capture probe and comprise nucleotide sequences that are complementary to a portion of the 16s rDNA gene at a 3′ end. The template particle may comprise a plurality of distinct capture probes with nucleotide sequences that are complementary to different portions of 16s rDNA gene, thereby allowing sequences from across a significant portion of the 16s rDNA gene to be captured and profiled. To design the capture probes, one must know have sequence information for the 16s rDNA gene. Accordingly, one database useful with the present invention is Greengenes, which is a web application that provides access to 16S rDNA gene sequence alignment for browsing, blasting, probing, and downloading. The database provides full-length small-subunit (SSU) rDNA gene sequences from public submissions of archaeal and bacterial 16S rDNA sequences. It provides taxonomic placement of unclassified environmental sequences using multiple published taxonomies for each record, multiple standard alignments, and uniform sequence-associated information curated from GenBank records. See DeSantis et al., 2006, Applied and Environmental Microbiology 72:5069-72.

Binding may involve incubating the partitioned sample at a temperature between 55° Celsius and 35° Celsius for approximately 1 hour. Under these conditions, any microbial nucleic acids present in the droplets hybridize with the nucleotide sequences of the capture probes via complementary base pairing. Preferably, the microbial nucleic acid hybridizes at a 3′ end of the capture probe.

After binding, the microbial nucleic acid is amplified. Preferably, the microbial nucleic acid is amplified inside the droplet. Alternatively, the droplet may be lysed, and microbial nucleic acid bound with the template particle may be recovered and amplified. Various methods or techniques can be used to amplify the microbial nucleic acid, for example, as discussed in WO 2019/139650, and WO 2017/031125, which are both incorporated by reference. Preferably, amplifying is accomplished by PCR to generate a copy of the microbial nucleic acid, i.e., an amplicon.

Amplicons from the target microbial nucleic acid amplification can be barcoded and sequenced.

The sequence reads may be analyzed to identify microbes. Various strategies for the alignment and assembly of sequence reads, including the assembly of sequence reads into contigs, are described in detail in U.S. Pat. No. 8,209,130, incorporated herein by reference. Strategies may include (i) assembling reads into contigs and aligning the contigs to a reference; (ii) aligning individual reads to the reference; or (iv) other strategies known to be developed or known in the art. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

PCR amplification involves the selective amplification of DNA or RNA targets using the polymerase chain reaction. During PCR, short single-stranded synthetic oligonucleotides or primers may be extended on a target template using repeated cycles of heat denaturation, primer annealing, and primer extension. In preferred systems and methods, primers used to amplify target nucleic acids in droplets are specific for a sequence in the target nucleic acids. According to other embodiments, a mixture of random synthetic primers may be included.

Preferably, primers are added to the mixture before portioning the sample into. Alternatively, the primers may be stored inside a compartment on the template particle and released into the droplet via an external stimulus, such as heat. The primers bind with a target nucleic acid, thereby priming the target nucleic acid for amplification by a DNA polymerase.

In some embodiments, a primer used in an amplification reaction can be attached to a surface of a template particle. In some embodiments, a surface of the template particle can comprise a plurality of primers.

In other preferred embodiments, some primers are not attached to the template particles and rather are included in an aqueous fluid and are segregated into the monodisperse droplets upon shearing the mixture. In other embodiments, some primers are delivered into the droplets via compartments within the particle templates.

In some aspects, non-PCR based DNA amplification techniques may be used. For example, in some instances multiple displacement amplification (MDA) methods can be used to amplify target nucleic acids inside droplets. For example, see U.S. Pat. No. 6,124,120, which is incorporated by reference. MDA amplification may have advantages over the PCR-based methods since MDA amplification can be carried out under isothermal conditions. No thermal cycling is needed because the polymerase at the head of an elongating strand (or a compatible strand-displacement protein) will displace, and thereby make available for hybridization, the strand ahead of it. Other advantages of multiple strand displacement amplification include the ability to amplify very long nucleic acid segments (on the order of 50 kilobases) and rapid amplification of shorter segments (10 kilobases or less). In multiple strand displacement amplification, single priming events at unintended sites will not lead to artefactual amplification at these sites (since amplification at the intended site will quickly outstrip the single strand replication at the unintended site).

In some instances, amplifying may occur by nonspecific amplification methods. For example, primers containing random sequences may be used. In other instances, sequence-specific amplification methods are used. Therefore, in some embodiments, amplification reactions include one or more primers. For example, in some embodiments, each droplet may include at least 20 primer pairs. In some embodiments, each droplet may comprise at least 50 primer pairs. In some embodiment, each droplet may comprise at least 200 primer pairs. In some embodiments, each droplet may comprise at least 500 primer pairs.

In preferred embodiments, amplifying is performed by PCR in the presence of a fluorophore in order to detect the target nucleic acid and/or the resulting amplicons.

In certain aspects, labels, such as intercalating dyes are incorporated into amplicons. In some embodiments, the droplets are lysed to release the fluorescently labeled amplicons prior to detection. After lysing the droplets, the sample may undergo one or more washing steps to rid the sample of fluorophores not incorporated inside DNA and thus make it easier to detect the presence of amplicons. At this stage, the amplicon may still be attached to the template particle. Any amplicons present in the sample will emit a fluorescent signal on account of the fluorophores. Because the amplicons are copies of microbial nucleic acids, the fluorescent signal is indicative of target nucleic acids present in the sample.

Samples positive for target nucleic acids may be processed for sequencing. In some embodiments, this involves amplifying the bead-bound amplicons. Amplifying bead-bound amplicons may be performed with primers that include sequencing primers.

In some embodiments, amplified target nucleic acids may be analyzed by sequencing, which may be performed by any method known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Sequencing generates sequence reads, which must be processed. The conventional pipeline for processing sequencing data includes generating FASTQ-format files that contain reads sequenced from a next generation sequencing platform, and aligning these reads to an annotated reference genome. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

The sequence reads may be aligned to one or more references to identify a microbe from which a microbial target DNA in a sample originated. Accordingly, the one or more references may include nucleotide sequences from known microbes. Matching the sequence reads from the microbial nucleic acids with the nucleotide sequences of known microbes is useful to determine the identity of the microbe in the patient based on the sequenced microbial nucleic acids. As such, in preferred embodiments, methods of the include sequencing amplicons, i.e., the copies of microbial nucleic acids from the sample, to produce a plurality of sequence reads. The amplicons may optionally be amplified prior to sequencing. Sequencing may be performed with any known sequencer.

Analyzing the sequence reads may include aligning them to one or more references of known microbes. This may be performed using a computer program. For example, analyzing the sequence reads may be performed with using the Basic Local Alignment Search Tool (BLAST), developed by National Center for Biotechnology Information. Methods of the invention may include analyzing the sequence reads to identify the species of microbe present in the patient. Thus, another database useful with the present invention is Greengenes, which is a web application that provides access to 16S rRNA gene sequence alignment for browsing, blasting, probing, and downloading.

Unique molecule identifiers (UMIs) are a type of barcode that may be provided to nucleic acid molecules in a sample to make each nucleic acid molecule, together with its barcode, unique, or nearly unique. This is accomplished by adding, e.g. by ligation, one or more UMIs to the end or ends of each nucleic acid molecule such that it is unlikely that any two previously identical nucleic acid molecules, together with their UMIs, have the same sequence. By selecting an appropriate number of UMIs, every nucleic acid molecule in the sample, together with its UMI, will be unique or nearly unique. One strategy for doing so is to provide to a sample of nucleic acid molecules a number of UMIs in excess of the number of starting nucleic acid molecules in the sample. By doing so, each starting nucleic molecule will be provided with different UMIs, therefore making each molecule together with its UMIs unique. However, the number of UMIs provided may be as few as the number of identical nucleic acid molecules in the original sample. For example, where no more than six nucleic acid molecules in a sample are likely to be identical, as few as six different UMIs may be provided, regardless of the number of starting nucleic acid molecules.

UMIs are advantageous in that they can be used to link amplicons to a single template nucleic acid and/or particle from which the amplicons were derived. After sequencing amplicons, sequence reads with identical UMIs may be considered to refer to the same starting nucleic acid molecule. In certain aspects, UMIs can help reduce amplification bias and correct for errors created during amplification, such as amplification bias or incorrect base pairing during amplification.

In certain aspects, sequence reads from amplicons can be aligned with a database of known sequences. This can help, for example, elucidate a mutation in a sample or identify a microbial nucleic acid.

A number of different databases may be helpful for obtaining reference sequences of microbes. Ensembl Genomes is a database useful for the present invention. Ensemble Genomes provides genome-scale data from non-vertebrate species. It complements the main Ensembl database (which focuses on vertebrates and model organisms) by providing genome data for bacteria, fungi, invertebrate metazoa, plants, and protists. The bacterial division of Ensembl contains all bacterial genomes that have been completely sequenced, annotated, and submitted to the International Nucleotide Sequence Database Collaboration (European Nucleotide Archive, GenBank, and the DNA Database of Japan). It contains more than 15,000 genomes. Ensembl allows manipulation, analysis, and visualization of genome data. Most Ensembl Genomes data is stored in My SQL relational databases and can be accessed by the Ensembl Pearl API, virtual machines or online. See Kersey et al., 2011, Nucleic Acids Research 40:D91-97.

The DNA Data Bank of Japan (DDBJ) is another sequence database. The central DDBJ resource consists of public, open-access nucleotide sequence databases including raw sequence reads, assembly information and functional annotation. It exchanges its data with European Molecular Biology Laboratory at the European Bioinformatics Institute and with GenBank at the National Center for Biotechnology Information on a daily basis. See Kodama et al., 2012, Nucleic Acids Research 40:D38-42.

Several databases focus on a particular conserved gene, such as the 16S rDNA. For example, the EzTaxon-e database is a web-based tool for identifying prokaryotes based on 16S ribosomal RNA gene sequences. EzTaxon-e is an open access database containing sequences of type strains of prokaryotic species with validly published names. The database covers not only species within the formal nomenclatural system but also phylotypes that may represent species in nature. All sequences that are held in the EzTaxon-e database have been subjected to phylogenetic analysis, which has resulted in a complete hierarchical classification system. See Kim et al., 2012, International Journal of Systematic and Evolutionary Microbiology 62:716-21. The Ribosomal Database Project (RDP) is another database useful with the present invention. It provides aligned and annotated rRNA gene sequence data for bacterial and archaeal small subunit rRNA genes, as well as fungal large subunit rRNA genes. RDP provides tools for analysis of high-throughput data, including both single-stranded and paired-end reads. Most tools are available as open source packages for download. See Cole et al., 2014, Nucleic Acids Research 42:D633-42.

SILVA is another database providing comprehensive, quality checked, and regularly updated datasets of aligned small (16S/18S, SSU) and large subunit (23S/28S, LSU) ribosomal RNA (rRNA) sequences for bacteria, archaea and eukarya. It has an aligner tool called SINA (SILVA INcremental Aligner) that is able to accurately align sequences based on a curated SEED alignment. The aligner determines the next related sequences using an optimized Suffix Tree server. To find the optimal alignment for a new sequence up to 40 reference sequences are taken into account. The SINA tool is not useful for typing however. See Pruesse et al., 2012, Bioinformatics 28:1823-29; and Quast et al., Nucleic Acids Research 41:D590-96.

Another database useful with the present invention is Greengenes, which is a web application that provides access to 16S rRNA gene sequence alignment for browsing, blasting, probing, and downloading. The database provides full-length small-subunit (SSU) rRNA gene sequences from public submissions of archaeal and bacterial 16S rDNA sequences. It provides taxonomic placement of unclassified environmental sequences using multiple published taxonomies for each record, multiple standard alignments, and uniform sequence-associated information curated from GenBank records. See DeSantis et al., 2006, Applied and Environmental Microbiology 72:5069-72.

Instruments and Systems

The present invention further provides systems that can, on a single instrument, include all the components necessary to conduct multiplex qPCR and/or dPCR assays, including thermocycle and signal detection components. The systems of the invention can perform multiplex qPCR and dPCR reactions using an emulsion of monodisperse droplets that each include a single template particle, a target nucleic acid, and reagents necessary for PCR amplification. Further, these reactions can be performed using a simple fluidic cartridge, obviating the need for complex microfluidics. Since the systems are unconstrained from the costs and throughput issues caused by complex fluidics, they provide a low cost and scalable modality for conducting multiplex amplification-based detection assays.

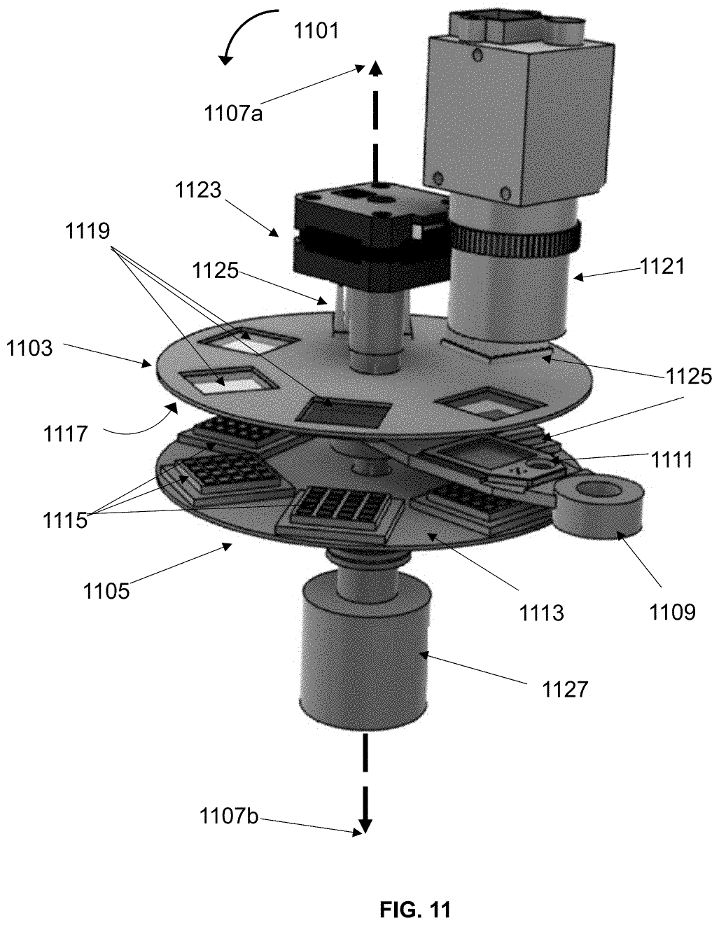

FIG. 11 provides a schematic of an exemplary system 1101 of the invention. The system 1101 includes a first rotating stage 1103 and a second rotating stage 1105. Each stage is disposed around a central axis illustrated by vectors 1107a and 1107b. Between the stages (1103, 1105) is a cartridge holder 1109, which can receive a fluidic sample cartridge 1111.

Each stage (1103, 1105) includes a surface perpendicular to the central axis that faces the cartridge holder 1109, which is referred to herein as a working surface. The working surface 1113 of the second stage 1105 can include one or more illumination zones 1115 arranged on the perimeter of the second stage 1105.

The working surface 1117 of the first rotating stage 1103 can include one or more optical elements 1119, such as optical filters and lenses, arranged around the perimeter of the first stage 1103. As shown, these optical elements may pass through the first stage.

The system 1101 can also include one or more thermal elements 1125 on each stage (1103, 1105). As shown, the thermal elements 1125 can traverse the thickness of its stage, such that a portion of each element can interface with the fluidic sample cartridge 1111.

The system can further include an imaging subsystem 1121, which may include, for example, a camera. The system 101 further includes a means 1123, such as a motor, for rotating the stages around the central axis. The system also includes a clamping system 1127, which can be used to change and control the distances between the second stage 1105, the sample cartridge holder 109, and/or the first stage 1103 in a direction along the central axis.

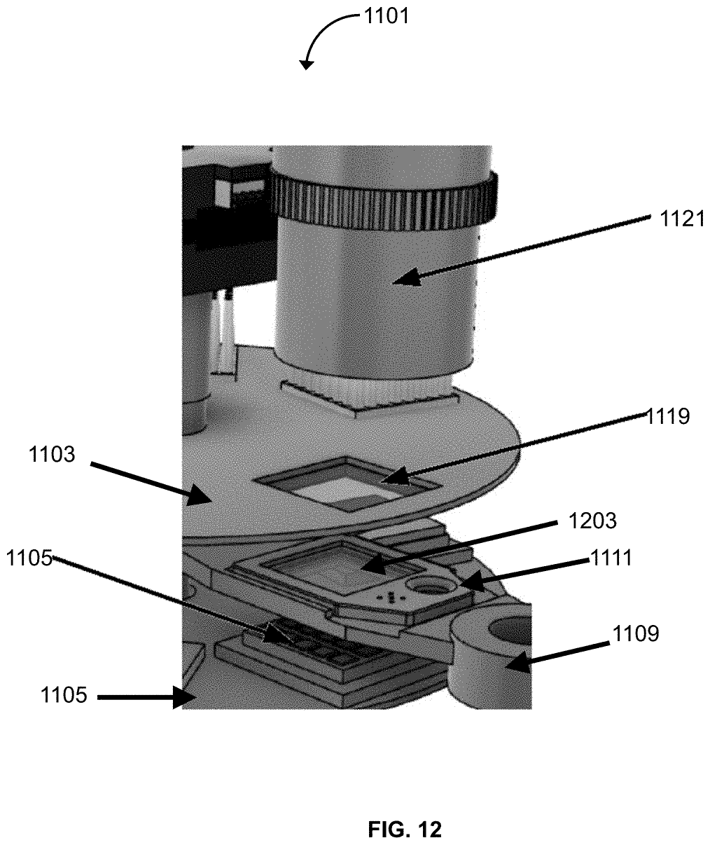

FIG. 12 provides a closeup view of the exemplary system 1101. As shown, the first rotating stage 1103 and second rotating stage 1105 can rotate around the central axis to align an optical element 1119, an illumination zone or source 1115, the optical subsystem 1115, and a sample area 1203 of the sample cartridge 1111 on the cartridge holder 109.

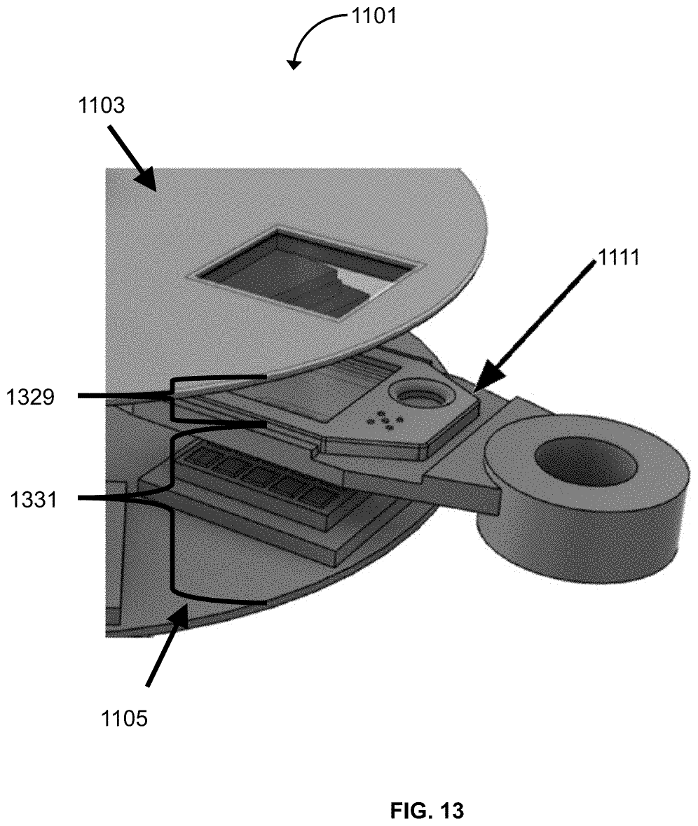

FIG. 13 provides different closeup view of exemplary system 1101. As shown, the first rotating stage 1103 and the sample cartridge 1111 are separated by a distance 1329 in the direction of the central axis. Similarly, the second rotating stage 1105 and the sample cartridge 1111 are separated by a distance 1331 in the direction of the central axis. These distances are adjusted and controlled via the clamping system 1127.

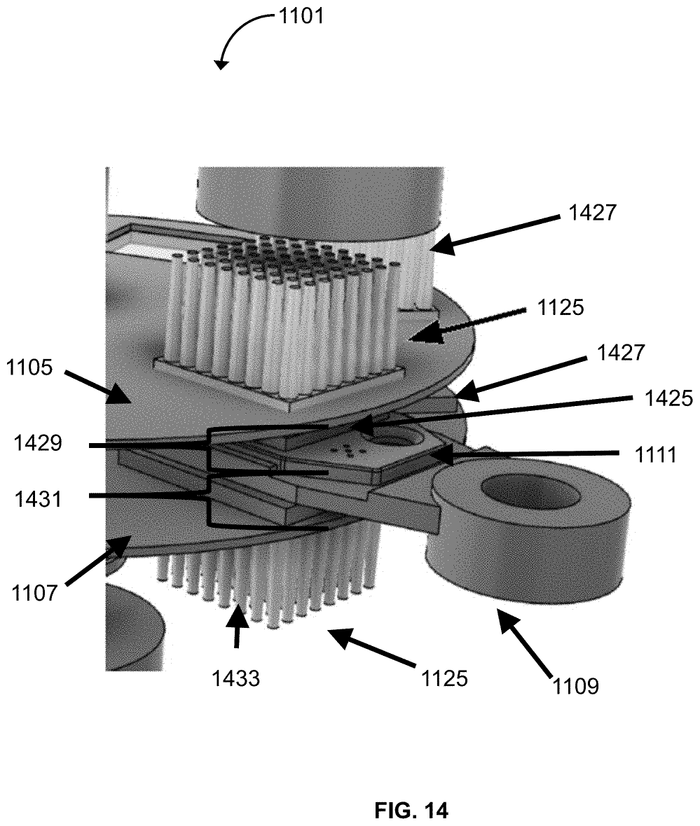

FIG. 14 provides another closeup view of exemplary system 1101. In this view, the first and second rotating stages have been rotated around the central axis to align thermal elements 1125 with the sample cartridge 1111. Rotating the thermal elements 1125 into alignment with the cartridge 1111 concurrently displaces the optical element 1119 and illumination zone 1115 from alignment with the cartridge.

Also, as shown in FIG. 14, the clamping element 1127 has moved the first and second stages along the central axis such that the distance 1429 between the first stage and sample cartridge and the distance 1431 between the second stage and the sample cartridge has been reduced. By reducing this distance, a portion 1425 of each aligned thermal element 1125 seats on or within the sample area 1203 of the sample cartridge 1111. As such, the thermal elements are able to heat/cool the sample from two sides. Since the sample volume within the cartridge is small compared to the areas heated by the two thermal elements, the temperature of the sample can be quickly be brought into thermal equilibrium with the thermal elements.

Further, as shown in FIG. 14, the first and second stages may also include additional thermal elements 1427. The additional thermal elements may be set at a different temperature setting relative to the first set of the thermal elements. Thus, in certain aspects, the system 1101 can quickly rotate the stages and align the sample cartridge with either the thermal elements 1125 or the additional thermal elements 1427. In this way, the system can quickly alternate the temperature of the sample as desired. Thus, the system can be used as a thermocycler for numerous bioassays, such as those using PCR.

The thermal elements (1125, 1427) can be, for example, thermoelectric devices, such as Peltier devices. Preferably, the thermal elements are solid-state Peltier devices. As shown in FIG. 14, the thermal elements include a portion 1425 on the working surfaces of the rotating stages (1105, 1107). This portion 1425 of each thermal element can interface with the cartridge 1111 when brought into alignment to heat or cool a sample in the cartridge. In certain aspects, the thermal elements can also heat/cool the cartridge 1111 itself to prevent any hot/cold spots on the cartridge or in the sample, which could otherwise cause evaporation or condensation in the sample. In certain aspects, the thermal elements can include a second portion 1433, which can be, for example, a heat sink.

The thermal elements (1125, 1427) can be made from, or include portions made from a heat conductive material such as, for example, aluminum, aluminum oxides, bismuth, bismuth telluride, bismuth-antimony alloys lead telluride, silicon germanium, silver, copper and/or other metals and/or alloys of any thereof. In certain aspects, the thermal elements include non- or moderately-conductive materials, such as certain ceramics, silicon, carbon-based materials, and the like. In certain aspects, heat conductive materials are interposed or disposed behind another material, such as a ceramic, which can assist to evenly distributes the thermal effect of the thermal elements on the sample.

In certain aspects, each individual thermal element (1125, 1427) includes a number of discrete zones. The temperature of each zone can be independently controlled such that different samples or areas of a sample in the cartridge 1111 can be brought to a different temperature.

In certain aspects, each individual thermal element (1125, 1427) includes one or more sensor (e.g., a thermocouple) that monitors the temperature of each element. Alternatively or additionally, the system includes one or more sensor to measure the temperature of the cartridge and/or sample in the cartridge. In either iteration, the sensors provide system with the ability to monitor and control the thermal elements to maintain constant temperatures or change temperatures as required.

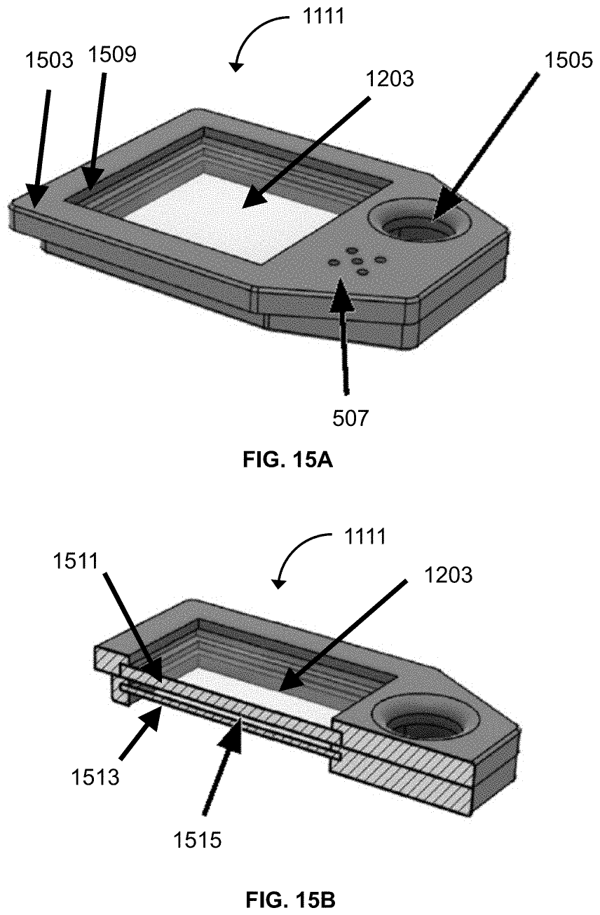

FIG. 15A provides a schematic of an exemplary fluidic cartridge or chip 1111 used with exemplary system 1101. A shown, the fluidic cartridge comprises a chassis or body 1503 that surrounds a sample area 1203. The cartridge may also include, for example, an inlet port 1505 and one or more vents 1507.

FIG. 15B provides a cutaway view of the exemplary fluidic cartridge or chip 1111. As shown, the sample area 1203 includes an upper substrate 1511 and a lower substrate 1513. In certain aspects, the area between the substrates 1515 defines the volume of the sample area 1203. Preferably this is a known and defined volume.

As shown in FIGS. 15A-15B, the sample area 1203 can be recessed relative to the upper surface of the chassis 1503. As shown in FIG. 14, the sample area can be dimensioned such that a portion of the thermal elements 1425 can be seated on the sample area 1203 via translation using the clamping system 1127 when the elements are brought into alignment with the cartridge 1111. This ensures that the entire surface of each substrate is in simultaneous and even contact with the thermal elements to provide consistent heating/cooling across the sample. Moreover, as shown in FIG. 15A, a portion 1509 of the chassis/body 1503 adjacent to the sample area can be contacted by the seated portion 1425 of the thermal element 1125. Thus, the thermal element 1125 can heat/cool both the sample area and the cartridge 1111 itself to prevent any hot/cold spots on the cartridge or in the sample, which could otherwise cause evaporation or condensation in the sample.

In certain aspects, the substrates are made from optically transmissible materials, such that electromagnetic radiation (e.g., light) can pass into and/or out of the sample area 1203. Preferably, the substrates are made from optically transmissible materials that are also thermally conductive. In certain aspects, one or both of the substrates is made from or includes, glass, silicon, silicon oxide, quartz, silicon nitride, polyethylene, polystyrene, glassy carbon, and epoxy polymers. Preferably, one or both of the substrates are made from an elastomeric polymer. Exemplary elastomeric polymers include, for example, polytetrafluoroethylene (PTFE) and polydimethylsiloxane (PDMS) polymers.

Preferably, the substrates are made from or include PDMS polymers. Non-limiting examples of PDMS polymers include those sold under the trademark Sylgard by Dow Chemical Co., Midland, Mich., including Sylgard 182, Sylgard 184, and Sylgard 186. Advantageously, silicone polymers, like PDMS, are inexpensive, readily available, and can be readily solidified using moderate heat. Further, silicone polymers are generally elastomeric, which facilities their use in forming small, detailed features, which can be integral in forming fluidic cartridges. Furthermore, PDMS and similar polymers can be specifically oxidized. The resulting oxidized structures include chemical groups that can cross-link to oxidized surfaces of many other polymeric and non-polymeric materials. This crosslinking behavior can be used to irreversibly seal the substrates to the cartridge without the need to use adhesives or other sealing means. Relevant oxidation and sealing techniques include those found Duffy et al., “Rapid Prototyping of Microfluidic Systems and Polydimethylsiloxane,” Anal. Chem., 70:474-480, 1998, which is incorporated by reference. Oxidized silicone polymers are also, generally, more hydrophilic than other elastomeric polymers. Thus, when used to form the sample facing surfaces of the substrates, or portions thereof, oxidized silicone polymers surface are readily filled and wetted with aqueous solutions.

Such a cartridge arrangement can be especially beneficial when using a sample of PIP encapsulated monodispersed droplets in, for example, a dPCR assay. The dimensions and resulting volume of the sample area in the cartridge have a wide area, but low volume, such that when introduced into a sample, monodisperse droplets organize into a monolayer between the substrates. Given the small volume of the cartridge, the droplets can be in contact with one or more both of the substrates. The monodispersed droplets abutting the substrates can thus be heated/cooled evenly, across the surface of the substrates using the thermal elements. Unlike a traditional PCR tube heated in a thermal block, using the cartridge as described, most if not all of the sample is at an equal distance from the thermal elements. Thus, heating can proceed evenly across the entire sample, thereby permitting faster and more accurate thermal cycling. Furthermore, the entirety of the sample (now a monolayer) can be imaged through the optically clear substrates. As the sample is distributed evenly across the substrates, excitation light can be evenly transmitted to the sample to, for example, excite fluorescent reporters. Detectable signals, such as those produced from fluorescent reporters, can in turn be read from the droplets.

In certain aspects, the cartridge includes features that facilitate control of fluidic transport. Fluidic transport can include flowing reagents, samples, and other components into, out of, and around the cartridge. Fluidic control features include, for example, structural features (e.g., channels and partitions) physiochemical characteristics (hydrophobic and hydrophilic materials), mechanical features (e.g., valves) and/or other features that can exert a force (e.g., capillary and containing forces) on a fluid. In certain aspects, these features can be used to promote formation of a monolayer of monodisperse droplets and/or help position and index the monodisperse droplets in the sample area.

In certain aspects, the fluidic cartridge includes an inlet port 1505 through which sample, reagents, and other required materials can be introduced into the cartridge. In certain aspects, a cartridge of the invention includes a plurality of sample areas or a partitioned sample area. Samples and reagents can be introduced into each sample area/partition using separate inlet ports 505, or using a single inlet port that can access each sample area/partition.

In certain aspects, the cartridge 1111 also includes one or more outlet port or module. An outlet port or module is an area of the cartridge that collects or dispenses molecules, cells, small molecules or particles for recovery or as waste. The outlet module can include a collection module to collect portions of the sample for recovery and/or a waste module to remove assay components as needed. The collection and/or waste module can be, for example, a well or reservoir for collecting particles released from a PIP droplet. droplets detected to have a specific predetermined characteristic in the detection module. The outlet port may contain branch channels or outlet channels for connection to a collection module or waste module. A device can contain more than one outlet module. In certain aspects, the outlet and inlet port(s) are the same port.

In certain aspects, the cartridge is flow cell. In certain aspects, fluid that includes, for example, sample or reagents can be injected into the inlet port. Fluid can be continually introduced as fluid flows out, for example, using an outlet port. This can allow the systems of the invention to, for example, perform flow-based detection methods and conduct assays with a series of washing steps without removing the cartridge from the system.

The cartridge can also include one or more vents or valves 1507. The vents 1507 can be used to ensure that when a sample is introduced into the cartridge via the inlet port, any air or other undesired material is pushed out of the cartridge. In certain aspects, the vents can interface with a pump or vacuum to move fluid into, out of, and around the cartridge.

In certain aspects, the substrates and/or other portions of the cartridge in fluidic contact can include a coating that prevents samples and/or reagents from adhering to the cartridge in an undesired fashion. For example, the sample-facing surfaces of the substrate may include an anti-wetting or blocking agent. Exemplary coatings include, for example, proteins that prevent adhesion of a biological/chemical sample, Teflon®, BSA, PEG-silane, fluorosilane, one or more cyclized transparent optical polymer, and other similar treatments known in the art.

In certain aspects, samples assayed using the systems of the invention include the use of magnetic components, such as magnetic or paramagnetic beads. These beads, and any molecules bound to them (e.g., target nucleic acids and amplicons), can be moved and directed using a magnetic system. In certain aspects, the system includes a magnetic system in the sample holder. In certain aspects, the system can use the magnetic system to position the magnetic beads in the cartridge. Samples attached to the magnetic beads can be recovered from the cartridge. In certain aspects, magnetic or paramagnetic beads used with the system are template particles to form monodisperse droplets as described herein. The system may be used to run an amplification assay (e.g., PCR or dPCR) of nucleic acids in a monodisperse droplet. Amplicons of the reaction may be bound to a magnetic PIP template particle and recovered after lysis of the droplets.

Returning to FIG. 11, in certain aspects, the systems of the invention include an imaging subsystem 1121, one or more illumination zones 1115 and one or more optical elements 1119. Using these components, in conjunction with the thermal elements and the cartridge, the exemplary system can be used to perform a number PCR amplification-based detection assays, such as digital PCR (dPCR) and quantitative PCR (qPCR). These assays are often performed using optically detectable labels, such as fluorescent dyes, which produce optical signals. The systems of the invention can thus include an imaging subsystem 1121 to parse and detect these optical signals.

However, in certain aspects, the systems of the invention can employ other types of signals, detection means and modalities. For example, the systems of the invention can include an electronic sensor to detect and measure electronic fields or other electrical characteristics, e.g., capacitance and inductance. Other modalities that can be used include, for example, infrared signals, ultraviolet signals, radioactivity, mass, density, volume, viscosity, pH, temperature, ion concentration, and the like. Advantageously, given the rotating nature of the stages, the required components to detect and activate these signals in a sample can be included on the rotating stages. Thus, several distinct characteristics of a sample can be measured by rotating the stages and aligning the sample cartridge with the appropriate components.

In certain aspects, the imaging subsystem 1121 includes one or more optical signal detectors. Exemplary optical signal detectors include, for example, cameras. The systems of the invention may include more than one camera/detector, but use only one or some of them for any particular assay. The imaging subsystem may include a means for moving the detector(s). In certain aspects, the detector can be moved parallel to the central axis and/or perpendicular to the central axis. Alternatively or additionally, the detector can be yawed or tilted relative to the central axis.

In the preferred case of fluorescent labelling, various lenses, illumination sources, excitation light sources, and filters may be used. Imaging modules may include any device capable of producing a digital image of the detectably labeled target cells, molecules, viruses, or microbes in a solution or pulled to a detection surface in a well or testing device. Imaging modules may include, for example, CCD cameras, CMOS cameras, line scan cameras, CMOS avalanche photodiodes (APD's), photodiode arrays, photomultiplier tube arrays, or other types of digital imaging detectors.

Preferably, the imaging subsystem 1121 includes a digital imager to detect fluorescent signals. The imaging subsystem may thus include any device that can detect fluorescent signals to produce a digital image. Digital imagers include, for example, CCD cameras, CMOS cameras, line scan cameras, CMOS avalanche photodiodes (APD's), photodiode arrays, photomultiplier tube arrays, or other types of digital imaging detectors. In certain aspects, a digital imaging detector includes an array of independent photosensitive pixel elements. Exemplary arrays include linear arrays, two-dimensional arrays, and prism arrays. Pixel elements lying in the path of emission light from a target in a sample detect emitted photons that impinge on the pixel elements to produce a resulting image of the sample. The imaging subsystem may also include various lenses, prisms, objectives, mirrors, filters, baffles, slots, collimators, light dispersal elements, light blocking components, light sources, and other light directing components.

To increase the flexibility of the exemplary system, one or more illumination zones 1115 can be disposed along the perimeter of the of the working surface 1113 of the second stage 1105. Alternatively or additionally, the exemplary system can include illumination zones on the working surface 1117 of the first stage. For example, an illumination zone(s) or source(s) can surround, or be incorporated with, an optical element 1119 on the first stage 1103. Alternatively, the first stage may not include an optical element, and simply have a cutout in the first stage around which an illumination zone(s) or source(s) is located.

Each illumination zone includes one or more illumination source. Exemplary light sources include an incandescent lamp, a gas discharge lamp, a light emitting diode (LED), an organic LED (OLED), a diode laser bar, a laser, a diode laser, or any other suitable light source. In certain aspects, an illumination zone includes more than one light source. A light source or groups of light sources in a single illumination zone can be independently or individually addressable, such that each source or group can be controlled separately. In certain aspects, light sources can be controlled by channel. For example, if an illumination zone includes a number of red, green, blue (RGB) diodes, the relative intensity of each color (channel) can be controlled, but is the same across all diodes of the array.

In certain aspects, an illumination zone only transmits light of a specific wavelength or range of wavelengths. In such instances, the system may include a number of different illumination zones, which each transmit light of a spectrally distinct wavelength of range. Alternatively or additionally, the system includes one or more illumination zones that can transmit light across multiple wavelengths or wavelength ranges.

In certain aspects, an illumination zone or source transmits light to a sample in the fluidic cartridge to stimulate a reagent or sample in the fluidic cartridge. This stimulation light can, for example, be transmitted to a fluorescent reporter in the fluidic cartridge. Illuminating the reporter with the stimulating light energizes the reporter, causing it to emit light at a wavelength different than the stimulation light. As the emitted light is spectrally distinct from the simulating light, it can be distinguished and detected by the imaging subsystem.

The systems of the invention can illuminate samples in a variety of manners. Exemplary illumination modalities include, for example, using trans-illumination, epi-illumination, edge-illumination, total internal reflection fluorescence, and slimfield illumination.

Trans-illumination generally involves transmitting light, including stimulating light, through a sample. Light can be attenuated in the sample and propagated to the detector or used to excite a reporter. By using a fluidic cartridge with optically transmissible substrates, light can be transmitted through the sample to employ trans-illumination. Advantageously, trans-illumination can be used with a light source displaced (or moved into position using the clamping device) in close proximity to the sample. This assures, that relative to other illumination modalities, highly intense light can be transmitted to the sample.

In contrast, epi-illumination generally involves transmitting light to a sample, which is then reflected in the sample and propagated to the detector or to stimulate a reporter. Epi-illumination is particularly useful in samples that are not translucent or transparent, which often cannot be imaged using trans-illumination. In contrast to trans-illumination, epi-illumination generally requires that a light source is displaced further from the sample. Advantageously, due to the flexibility conferred by the clamping system, the systems of the invention can use both trans-illumination and epi-illumination by adjusting the distance the sample cartridge is to a light source.

The system of the invention can also provide total internal reflection fluorescence (TIRF) illumination, in which illumination light is transmitted at a steep angle of incidence onto a surface, which creates an evanescent wave along the surface (e.g., the fluidic cartridge substrate) at an interface of reflection. This causes illumination a thin region of the sample adjacent to the surface. TIRF can illuminate a region as thin as a few hundred nanometers. Advantageously, this can reduce or eliminate autofluorescence of unwanted materials in the sample or cartridge. Slimfield illumination is similar to TIRF illumination, however, the resulting illumination covers a narrower area along the surface of the sample, while also illuminating an area further away from the surface relative to TIRF.

In order to control and direct light being transmitted to and/or from a sample in the sample cartridge, the systems of the invention can include optical elements 1119 arranged around the perimeter of the first stage 1103. Optical elements 1119 can include various light directing components as required for a particular assay, illumination type, reporter, sample type, and the like. Light directing components include, for example, various lenses, prisms, objectives, mirrors, filters, baffles, slots, light dispersal components, light blocking components, light sources, and the like. In certain aspects, the systems of the invention include light directing components in optical elements 1119 on the first stage as well as in any of the light zone(s) 1115, the imaging subsystem 1121, and the fluidic cartridge 1111. Alternatively, the systems of the invention do not include optical elements on the first stage, and instead have a cutout on the first stage, which can be aligned with the imaging subsystem and the sample cartridge. In such cases, light directing components can be incorporated with one or more of the light zone(s), imaging subsystem, and the cartridge.

The systems of the invention can use a variety of methods, and the associated hardware arrangements, to detect and distinguish emission light from fluorescent reporters in a sample. Exemplary methods and optical components for detecting and distinguishing emission light include those as provided in Wang et al. “Multiplexed Optical Imaging of Tumor-Directed Nanoparticles: A Review of Imaging Systems and Approaches”, Nanotheranostics, 2017, Vol. 1, p. 369-387, which is incorporated herein by reference.

In certain aspects, the systems of the invention can perform scanning-based and/or wide-field imaging and detection.

In scanning-based imaging, such as raster scanning, a collimated or focused beam of light (often a laser) is scanned across a specimen. When compared with wide-field imaging, scanning-based imaging provides improved spectral resolution by collecting a highly resolved spectrum at each line or point of pixels on the sample. Generally, the focused beam of light has a distinct shape, such as a line or spot. This light can be used to directly image a sample and/or excite a fluorescent reporter. In certain aspects, the systems of the invention move the beam of light across by steering the beam itself using, for example, a scanning mirror. Alternatively or additionally, the sample itself can be moved. Thus, the presently disclosed systems can include a sample cartridge holder that translates that cartridge in one or more of the x, y, and z directions relative to an illumination source and/or the imaging subsystem.

The system can employ a variety of optical detectors for use in scanning-based imaging, including, for example, photomultiplier tubes (PMT), avalanche photodiodes (APD), charged coupled devices (CCD), intensified CCDs (ICCD), electron-multiplying CCDs (EMCCD), and scientific complementary metal—oxide semiconductor (sCMOS) arrays.

In wide-field imaging, an entire sample or region of a sample is illuminated. Emitted light from an array of points/pixels within the illuminated region is detected using a two-dimensional array. Unlike scanning-based modalities, there is no need to move the sample/illumination beam. Moreover, because an entire area or sample is illuminated at once, wide-field imaging is often simpler and cheaper to implement.

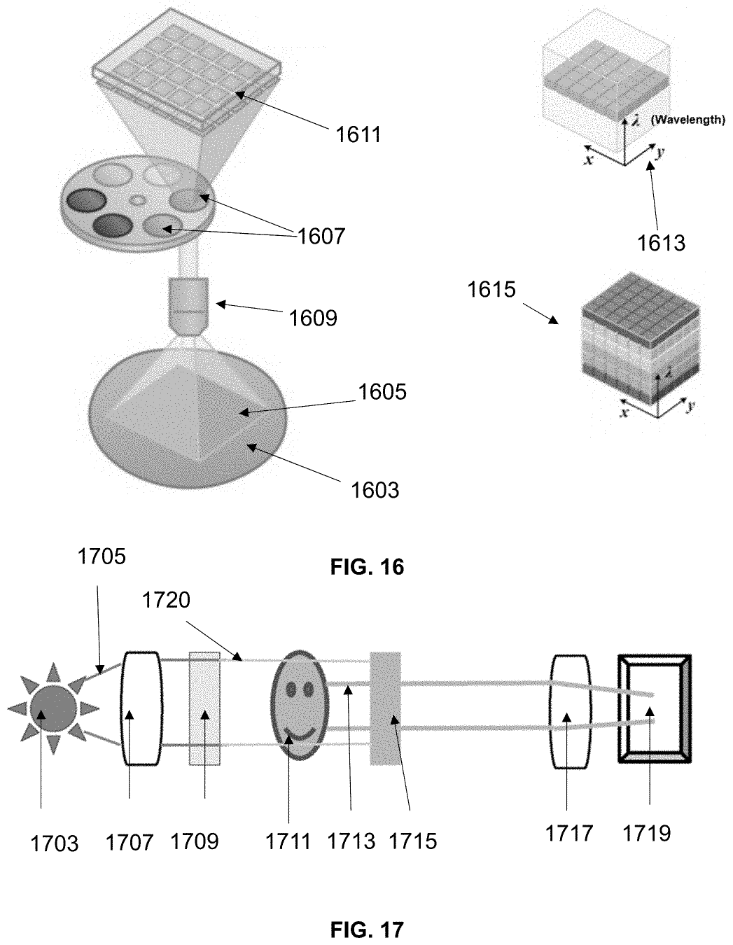

In certain aspects, the systems of the invention employ widefield wavelength scanning. FIG. 16 provides a generalized schematic of widefield wavelength scanning. As shown, a region of a sample 1603 is illuminated. Preferably, the illumination light is uniform in wavelength and intensity across the illuminated region. Emission light 1605, which can be from excited fluorescent reporters in the illuminated region, is transmitted to a two-dimensional detector array 1611 and mapped 1613 into a two-dimensional image. The mapped, two-dimensional image 1613 is for a single wavelength, with the x and y axis representing the illuminated region of the sample (the λ axis represents the spectral wavelength). In certain aspects, multiple two-dimensional images are mapped for different wavelengths. These images can be combined to form a hyperspectral data cube 1615 (a “hypercube”) to find spatial areas of the sample that are emitting light at various spectral wavelengths.

In certain aspects, where a sample includes multiple, different fluorescent reporters, each with a distinct emission spectrum, the system includes a series of filters 1607. Each filter 1607 is specific to a certain reporter and its corresponding spectral emission. In certain aspects, the different filters are disposed on the first stage, and the stage is rotated to sequentially align the filters with the cartridge/detector in order to detect emission light from different reporters. Additionally, one or more light directing components 1609, e.g., lenses, can be used.

FIG. 17 provides a schematic of certain components used by systems of the invention to perform widefield wavelength scanning. A light source(s) 1703, e.g., an LED, such as found in an illumination zone as described herein, provides excitation light 1705. The systems may include a collection lens 1707, that collects and collimates the excitation light 1705.

In certain aspects, the system includes an excitation light filter 1709. The excitation light filter 1709 can, for example, filter any light other than that of a spectral wavelength or range of wavelengths to optimally excite a particular fluorescent reporter. As a result, only light 1720 with desired spectral properties transmits to the sample 1711. Also or alternatively, the excitation light (1705, 1720) is sufficiently spectrally distinct from the emission light 1713 of, for example, a fluorescent reporter such that it does not interfere with detection of the emission light.

The systems can include an emission light filter 1715. The emission light filter 1715, which can be, for example, a monochromator, allows emission light 1713 from an excited fluorescent reporter to pass through it. However, it blocks light of other wavelengths, such as the excitation light (1705, 1720), from passing through to the detector array 1719. Preferably, the emission light filter is, or is a component of, an optical element on the first stage as described herein. The systems may also include an imaging lens 1717 to focus the emission light 1713 onto the detector array 1719. The imaging lens can, for example, be a part of an imaging subsystem or an optical element on the first stage.

The systems of the invention can use widefield wavelength scanning to detect multiple, spectrally distinct fluorescent reporters from a sample.

Returning to FIG. 1, in certain aspects, the systems of the invention can use a separate emission light filter for each fluorescent reporter to be detected. The emission light filter for each separate fluorescent reporter can be, or is a component of, one of the optical elements 119 distributed on the perimeter of the first stage 103. Similarly, separate illumination zones 115 for each different fluorescent reporter can be distributed on the second stage 105. Each illumination zone 105 can provide spectrally optimized excitation light to cause a specific reporter to fluoresce. Thus, to detect different fluorescent reporters, the system sequentially rotates the stages to align the sample cartridge 111 and detection subsystem 121 with different emission light filters and illumination zones.

Alternatively or additionally, the systems of the invention use tunable filters. The wavelengths of light that are blocked or that pass through tunable filters can be changed or adjusted. Exemplary tunable filters include, for example, electronic tunable filters (e.g., liquid crystal tunable filters) and acousto-optic tunable filters.

As the emission light is filtered for every different reporter, in certain aspects, the detection subsystem 121 can include a monochrome detector/camera.

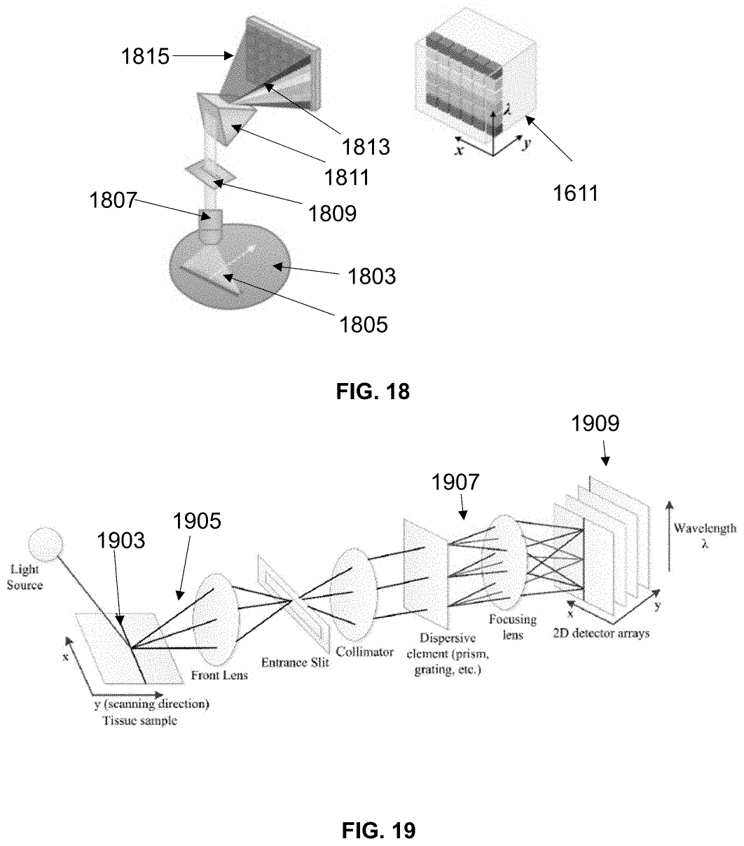

In certain aspects, the systems of the invention employ hyperspectral scanning. FIG. 18 provides a generalized schematic of hyperspectral scanning. As shown, a thin line of a sample 1803 is illuminated with broadband light, causing fluorescent reporters to produce emission light 1805. A lens 1807 directs the illumination light 1805 through a slit 1809. The light is collimated and then dispersed using a dispersive element 1811, such as a prism or grating. The dispersed light is detected by a two-dimensional array 1815 of a monochrome camera.

Raster scanning of the image can be accomplished by steering the thin line of illumination light across the sample, e.g., by using scanning mirrors. Alternatively or additionally, the camera and/or the sample are moved such that the line of light passes across the sample. Each detected line of emission light is mapped to a two-dimensional image 1817. The two dimensional image includes an x axis, which is the location on the sample along the illumination line and the λ dimension is the wavelength of light detected at each location (the y axis is a spatial axis on the sample perpendicular to the x axis). Since the two-dimensional image uses spectral wavelength as an axis, reporters with spectrally distinct emission spectra can be simultaneously detected, without the need to change filters, etc.

FIG. 19 provides a schematic of certain components used by systems of the invention to perform hyperspectral scanning. As shown, a broadband light source illuminates a narrow line 1903 of a sample. The resulting emission light from fluorescent reporters transmits from the sample to a front lens. The front lens directs the emission light through a slit, after which it is collimated. The collimated light enters a dispersive element, like a prism or grating. The resulting dispersed light 1907 enters a focus lens that directs it to a two-dimensional array to produce a two-dimensional image. As narrow bands of emission light are imaged across the sample, the resulting two-dimensional images are combined to form a hypercube.

In certain aspects, the systems of the invention employ sensor filter scanning. FIG. 18 provides a schematic of certain components used by systems of the invention to perform sensor filter scanning. As shown, a thin line of a sample 1803 is illuminated with broadband light, causing fluorescent reporters to produce emission light 1805. A lens 107 directs the illumination light 805 through a slit 809. The light is collimated and then dispersed using a dispersive element 811, such as a prism or grating. The dispersed light is detected by a two-dimensional array 815 of a monochrome camera.

Raster scanning of the image can be accomplished by steering the thin line of illumination light across the sample, e.g., by using scanning mirrors. Alternatively or additionally, the camera and/or the sample are moved such that the line of light passes across the sample. Each detected line of emission light is mapped to a two-dimensional image 817. The two dimensional image includes an x axis, which is the location on the sample along the illumination line and the λ dimension is the wavelength of light detected at each location (the y axis is a spatial axis on the sample perpendicular to the x axis). Since the two-dimensional image uses spectral wavelength as an axis, reporters with spectrally distinct emission spectra can be simultaneously detected, without the need to change filters, etc.

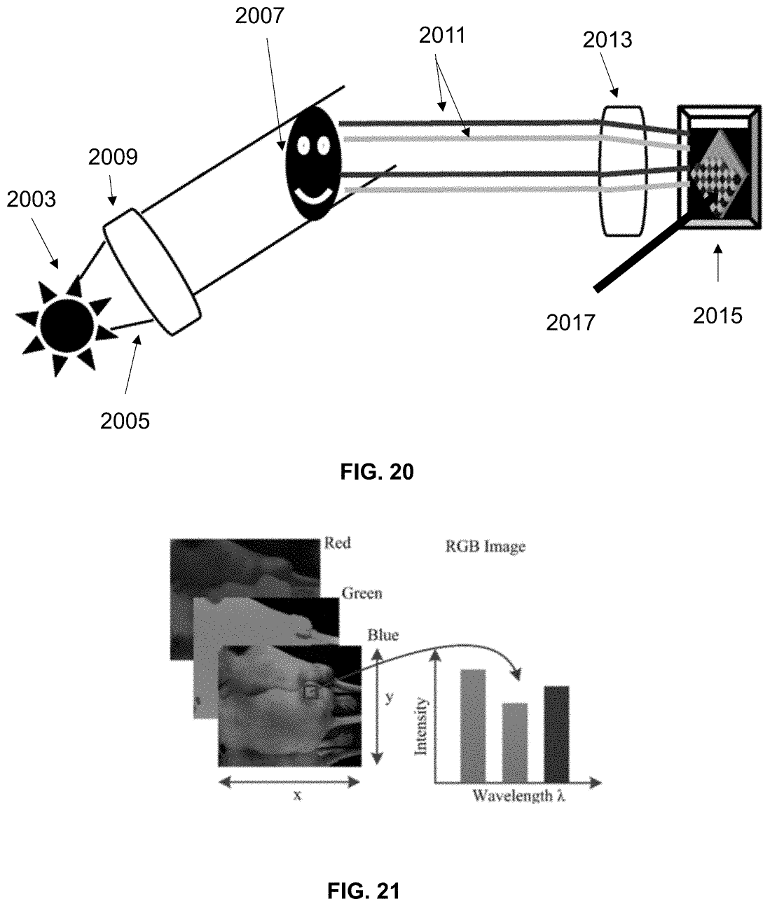

In certain aspects, systems of the invention employ sensor filer scanning and detection. FIG. 20 provides a schematic of certain components used by systems of the invention to perform sensor filter scanning.

As shown, a light source 2003 transmits uniform light 2005 to a sample, or a region of a sample 2007. In certain aspects, the light source provides white light to the sample. In such instances, the geometry and/or components of the system (e.g., baffles) prevent the illumination light from reaching the detector. Alternatively, the systems of the invention can use, for example, colored LEDs with filters to transmit uniform light of a desired range of wavelengths, which is spectrally distinct from emission light. Preferably, the systems of the invention include individually addressable RGB LEDs as the illumination source.

The systems may include a prism 2009 that directs the light to the sample 2007. Preferably, and as shown, the illumination light is transmitted to the sample at an oblique angle. The illumination light can include multiple different wavelengths, which can excite multiple different fluorescent reporters in the sample. As a result, spectrally distinct wavelengths of emission light 2011, each from a different reporter, are emitted from the sample. An imaging lens 2013 directs the emission light to a color camera 2015.

The color camera 2015 includes an array of pixels 2017. Each pixel includes a filter that only allows light of a particular wavelength range to pass through and be detected. Preferably, the array 2017 includes a mosaic, like a Bayer filter mosaic, of pixels that detect red light, pixels that detect blue light, and pixels that detect green light. In certain aspects, the array also includes pixels with near infra-red filters.

FIG. 21 shows the resulting images from light detected by the red, green, and blue pixel channels. The system can use demosaicing algorithms, and data from adjacent pixels in the array, to interpolate a set of intensity values for the red, blue, and green wavelengths of light in each pixel. These values can provide a signature to ascertain the spectrum of the emission light detected by a pixel, and thus at a location in the sample. This, in turn, can be used to determine the identity of a fluorescent reporter emitting the light.

Table 1, below provides the general components used in exemplary systems of the invention that employ either wavelength scanning, hyperspectral scanning, or sensor filter scanning. Table 2 provides the relative attributes of each time of system.

| | Wavelength | Hyperspectral | Sensor |
|---|---|---|---|
| Components | System | System | Filter System |
| Excitation LEDs | One per dye | Single broadband | Single RGB |
| | or single | | |
| | broadband | | |
| Illumination Lens | Yes | Yes | Yes |
| Excitation Filters | Yes | No | No |
| Emission Filters | Yes | Yes, two lenses, a | No |
| | | slit, and | |
| | | prism/grating | |
| Camera | Monochrome | Monochrome | Color camera with |
| | | | pixels having R, G, |
| | | | B, and NIR filters |
| Camera Imaging | Yes | Yes | Yes |
| Lens | | | |
| Motion/Raster | Yes | Yes | No |

TABLE 2

| | Wavelength | Hyperspectral | Sensor |
|---|---|---|---|
| Attributes | System | System | Filter System |
| Hardware | Medium | High | Low |
| Complexity | | | |
| Dye Flexibility | Low | High | High |
| Cost | High | Medium | Low |
| Signal to Background | Medium to High | High | Low |
| Image Processing | Medium | Low | High |
| Complexity | | | |

As shown in Table 2, wavelength systems have certain drawbacks relative to hyperspectral and sensor filter systems. The wavelength system requires different filters for each dye detected. This need to use multiple filters can increase the cost of the system. This expense is not due solely to the cost of the filters, but also because the system must include a means to change the filters, e.g., rotating stages, moveable camera, and/or moveable sample holder. Further, the systems may also require different sets of excitation LEDs for each dye. Moreover, because a unique filter is required for each spectrally distinct fluorescent dye, the system can be constrained in its ability to accommodate new or large numbers of different reporters.

Hyperspectral-based systems offer greater dye flexibility relative wavelength systems. This is because hyperspectral systems use spectral wavelength as an axis of the two-dimensional array, allowing the system to simultaneously detect a range of spectral emissions. This arrangement also provides the lowest image processing complexity. However, hyperspectral systems require complex optical elements and require means to steer the illumination light or move a fluidic cartridge to scan an entire sample.

Sensor filter systems offer the least complex hardware. They do not require moving parts, a highly complex arrangement of optical elements, or a series of different filters. Accordingly, sensor filter systems are the least expensive. Moreover, like hyperspectral systems, sensor filter systems can simultaneously detect multiple spectrums of emission light, lending them a high degree of dye flexibility. Sensor filter systems also provide the best signal to noise ratio of the systems, as the pixels have built in filter elements. The biggest drawback of sensor filter systems is their requirement for complex image processing, e.g., demosaicing algorithms that interpolate a set of intensity values for each pixel.

In certain aspects, the systems of the invention use one or more thermal elements, as described, to conduct a nucleic acid amplification reaction of one or more nucleic acids in a sample. The amplification reaction may be any amplification reaction known in the art that amplifies nucleic acid molecules, such as polymerase chain reaction, nested polymerase chain reaction, ligase chain reaction, ligase detection reaction, strand displacement amplification, transcription based amplification system, nucleic acid sequence-based amplification, rolling circle amplification, and hyper-branched rolling circle amplification. By using the opposing thermal elements and clamping system, the present system can perform a wide variety of nucleic acid amplification reactions. Further, by using additional sets of thermal elements, as described, temperatures in a sample cartridge can be quickly changed by rotating the stages to contact the sample with thermal elements pre-heated/cooled to a desired temperature.

In certain embodiments, the amplification reaction is the polymerase chain reaction. Polymerase chain reaction (PCR) increases the concentration of a target sequence of a nucleic acid in a mixture of DNA. The process for amplifying the target sequence generally includes introducing an excess of oligonucleotide primers to a DNA mixture containing a desired target sequence, followed by a precise sequence of thermal cycling in the presence of a DNA polymerase. The primers are complementary to their respective strands of the double stranded target sequence.

To effect amplification, primers are annealed to a complementary sequence in a target molecule. After annealing, the primers are extended with a polymerase to form a new pair of complementary strands. The steps of denaturation, primer annealing and polymerase extension can be repeated many times (i.e., denaturation, annealing and extension constitute one cycle; there can be numerous cycles) to obtain a high concentration of an amplified segment of a desired target sequence. The length of the amplified segment of the desired target sequence is determined by relative positions of the primers with respect to each other and by cycling parameters, and therefore, this length is a parameter that can be controlled using the system of the invention.

In certain aspects, the system can be used to amplifying nucleic acids isolated in a water-in-oil droplet, such as those that can be created using PIP encapsulation. Such droplets can be introduced into a cartridge as described herein. Sample droplets may be pre-mixed with a primer or primers, or the primer or primers may be added to the droplets. The droplets are thermal cycled using the thermal elements, resulting in amplification of the target nucleic acid in each droplet. Temperature profiles for thermal cycling can be adjusted and optimized as with any conventional DNA amplification by PCR.

In certain embodiments, the three temperatures are used for the amplification reaction. The three temperatures result in denaturation of double stranded nucleic acid (high temperature), annealing of primers (low temperature), and amplification of single stranded nucleic acid to produce double stranded nucleic acids (intermediate temperature). The temperatures fall within ranges well known in the art for conducting PCR reactions. See for example, Sambrook et al. (Molecular Cloning, A Laboratory Manual, 3^{rd} edition, Cold Spring Harbor Laboratory Press, Cold Spring Harbor, N.Y., 2001).

In certain embodiments, the three sequential temperatures are used in an amplification assay, which are approximately: 95° C. (T_{H}), 55° C. (T_{L}), 72° C. (T_{M}). In other embodiments two sequential temperatures are used, which are approximately: 95° C. (T_{H}) and 60° C. (T_{L}). Because the sample can be heated from both sides using two thermal elements, the temperature of the sample can be quickly changed. This is especially true when using a cartridge as described herein which allows the thermal elements contact a wide area of sample, from two sides, where the sample itself has a small volume. In certain aspects, two or more sets of thermal elements can be pre-heated to these desired temperatures, and the stages rotated to contact the cartridge with a set of thermal elements pre-heated/cooled to the desired temperature.

The presently disclosed systems can be used to perform a number of PCR-based assays, including quantitative PCR (qPCR), quantitative fluorescent PCR (QF-PCR), multiplex fluorescent PCR (MF-PCR), digital PCR (dPCR), single cell PCR, PCR-RFLP/real time-PCR-RFLP, hot start PCR, nested PCR, in situ polony PCR, in situ rolling circle amplification (RCA), bridge PCR, picotiter PCR, emulsion PCR and reverse transcriptase PCR (RT-PCR). Other suitable amplification methods that can be performed by the systems of the invention include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA).

In certain preferred aspects, the systems of the invention can be used to perform PCR-based assays to detect the presence of certain oligonucleotides and/or genes, e.g., oncogenes and other disease related genes. The inclusion of both thermal elements and signal detection elements makes this possible. Exemplary assays include, for example dPCR and qPCR assays.

In such assays, one or more primers specific to each target nucleic acid or gene of interest are reacted with the genome of each cell. These primers have sequences specific to the particular target, so that they will only hybridize and initiate PCR when they are complementary to the target. If the target of interest is present and the primer is a match, many copies of the target are created using PCR amplification. To determine whether a particular target is present in a droplet, the PCR products may be detected through an assay probing the liquid of the monodisperse droplet, such as by staining the solution with an intercalating dye, like SybrGreen or ethidium bromide, hybridizing the PCR products to a solid substrate, such as a bead (e.g., magnetic or fluorescent beads, such as Luminex beads), or detecting them through an intermolecular reaction, such as FRET or using fluorescent hydrolysis probes. These dyes, beads, and the like are each used to detect the presence or absence of nucleic acid amplification products, e.g., PCR products.

PCR- and real-time PCR-based detection methodologies have greatly improved the analysis of nucleic acids from both throughput and quantitative perspectives. Traditional PCR-based detection assays generally rely on end-point, and sometimes semi-quantitative, analysis of amplified DNA targets via agarose gel electrophoresis, real-time PCR (or qPCR) methods are most often used to quantify exponential amplification as the reaction progresses. Quantitative PCR reactions are monitored either using a variety of highly sequence specific fluorescent probe technologies, or by using non-specific DNA intercalating fluorogenic dyes.

Advantageously, because systems of the invention can include opposing thermal elements (one on each stage), the present systems can perform real-time PCR assays such as qPCR. When performing such assays, thermal elements on the second stage can be used to used to heat a sample in a cartridge to cause an amplification reaction in the sample. Concurrently, rather than aligning a thermal element on the first stage with the sample cartridge, optical elements, which may include one or more sources of illumination and the imaging subsystem can be aligned with the cartridge. Thus, while the aligned thermal element on the second stage provides the appropriate thermal inputs to cause the amplification reaction, the optical elements and the imaging subsystem can detect signals produced during the amplification reaction, e.g., from fluorescent reporters.

Digital PCR (dPCR) is an alternative quantitation method in which dilute samples are divided into many separate reactions in partitions, such as droplets formed by PIP encapsulation. The distribution from background of target DNA molecules among the reactions follows Poisson statistics at the terminal and/or limiting dilutions of target DNA. Generally, at a terminal dilution the vast majority of partitions contain either one or zero target DNA molecules. Ideally, at terminal dilution, the number of PCR positive reactions (PCR(+)) equals the number of template molecules originally present. At a limiting dilution, partitions include zero, one, and often more than one target nucleic acid following the Poisson distribution. At the limiting dilution, Poisson statistics are used to uncover the underlying amount of target DNA originally present in a sample.

To perform dPCR, the partitioned nucleic acids may be detected using labeled probes, such as hydrolysis probes. Exemplary hydrolysis probes include, for example, TaqMan probes produced by ThemorFisher Scientific. TaqMan probes include an oligonucleotide that binds to a specific sequence in the target nucleic acid. The probes include a detectable label, such a fluorescent dye, and a quencher. When attached to the probe, any signal produced by the fluorescent dye is quenched due the proximity of the dye to the quencher. During PCR amplification, exonuclease activity by a polymerase hydrolyzes the probe hybridized to the target nucleic acid. This, in turn, releases the fluorescent dye from the quencher, allowing it to produce a detectable signal indicative of a polymerase (amplification) reaction.

During imaging, partitions that produce a fluorescent signal from the released dyes are marked as a “1” or “0” (positive or negative for amplification), which informs the name “digital” PCR. Absolute quantification of the starting target nucleic acid in a sample can be calculated based on the ratio of PCR positive or negative partitions using Poisson statistics.

The principle advantage of digital compared to qPCR is that it avoids any need to interpret analog signals, i.e., real-time fluorescence versus temperature curves. Moreover, qPCR generally requires a standard curve, preferably from an on-chip standardization reaction to provide quantitative results. Digital PCR forgoes these complications, while still providing an absolute quantification.

Systems and instruments of the invention can be used to perform dPCR on nucleic acids from a sample isolated in monodisperse droplets. The systems and instruments can perform dPCR to determine the presence or quantity of one or more target nucleic acid in a sample. Thus, the systems of the invention can be used to perform diagnostic assays to quantify and/or detect the presence of a nucleic acid associated with a disease or other pathology. In certain aspects, the target nucleic acid is from a cell (e.g., circulating cells and/or circulating tumor cells), a virus, bacteria, or one or more genes of interest or genetic markers (e.g., oncogenes, or heterogeneous genes in a sample).

In certain aspects, the systems of the invention can be used to perform PCR-based detection assays, such as dPCR, without using physical partitions or droplets.

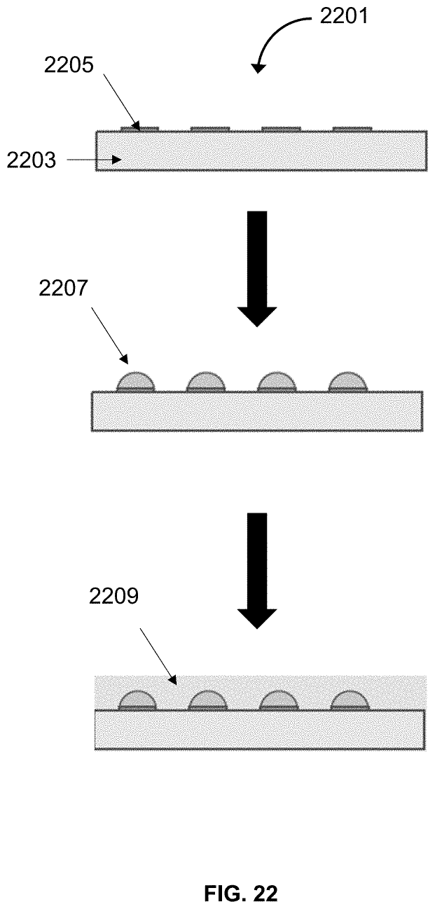

FIG. 22 provides an exemplary schematic 2201 of preparing a sample for amplification and detection using a system of the invention, without the use of physical partitions or droplets. As shown, a sample cartridge, such as that described herein, includes a substrate 2203 on which are a series of hydrophilic spots 2205 are disposed in a sample area of a known volume. In certain aspects, the areas between the spots are hydrophobic or super-hydrophobic. Each spot includes an attached primer, which can be used to amplify a target nucleic acid. An aqueous solution containing the target nucleic acid is flown into the sample area.

In certain aspects, the sample volume of the cartridge is low enough that when the cartridge is contacted with a vibrational energy, the aqueous solution coalesces into bubbles or bumps 2207 over the hydrophilic spots 2207, and thus away from the hydrophobic areas between the spots. Optionally, the sample can be dried, such that the bubbles evaporate into isolated aqueous bumps on the hydrophilic spots. In certain aspects, a non-aqueous fluid 2209, such as an oil, is introduced into the chip to cover the aqueous bumps to prevent further dehydration.

Inside the isolated aqueous areas, the primer attached to the substrate can be used to prime an amplification reaction. In certain aspects, the primers in each spot or regions of spots are known. Thus, more than one primer can be used, and the locations on the cartridge from which signals emanate function as spatial multiplexers. This allows a signal type of detectable label to detect the presence of an amplification reaction for a number of distinct target nucleic acids.

Thus, in certain aspects, the systems of the invention include an acoustic actuator. Preferably, the actuator is part of or is in contact with the sample cartridge holder. The actuator can provide acoustic energy to a sample (biological/chemical material) in a fluidic cartridge, which can mix and/or separate the sample. This may include, for example, distributing the sample hydrophilic areas by acoustic wave. The frequency of the acoustic wave should be fine-tuned so as not to cause any damage to samples. The biological effects of acoustic mixing have been well studied (e.g., in the ink-jet industry) and many published literatures also showed that piezoelectric microfluidic device can deliver intact biological payloads such as live microorganisms and DNA.

In certain aspects, the system of the invention can be used to amplify a nucleic acid sample, e.g., using PCR amplification, for uses off the presently disclosed system. For example, the systems of the invention can be used to perform PCR amplification of target nucleic acids. The amplification products can be recovered from the sample cartridge, e.g., through the use of a magnetic template particles. These amplicons can then be used to form a nucleic acid sequencing library to be sequenced in an off-system sequencer.

In certain aspects, systems and instruments include a computer, or are operably connected to a computer, which comprises a processor and a non-transitory, tangible memory and operable to schedule and control the components of the systems/instruments. The computer can include a user interface, including input/output devices (e.g., a monitor, keyboard, mouse, or touchscreen) for prompting and receiving information from the user and displaying results and status information. The computer can be used, for example, to direct the system to perform a specific type of assay. The computer can be connected to a network and operable to process test results and send to connected devices over the network.

The processor can be in communication with the instrument and the various motors and subsystems or stations thereon to, for example, rotate the stages, open/close the clamping system, and image a sample by controlling the illumination zones, optical elements, and imaging subsystem. Images recorded by the imaging subsystem can be sent to the non-transitory, tangible memory for analysis.

A processor refers to any device or system of devices that performs processing operations. A processor will generally include a chip, such as a single core or multi-core chip, to provide a central processing unit (CPU). A process may be provided by a chip from Intel or AMD. A processor may be any suitable processor such as the microprocessor sold under the trademark XEON E7 by Intel (Santa Clara, Calif.) or the microprocessor sold under the trademark OPTERON 6200 by AMD (Sunnyvale, Calif.).

Memory refers a device or system of devices that store data or instructions in a machine-readable format. Memory may include one or more sets of instructions (e.g., software) which, when executed by one or more of the processors of the disclosed computers can accomplish some or all of the methods or functions described herein. Preferably, the computer includes a non-transitory memory such as a solid state drive, flash drive, disk drive, hard drive, subscriber identity module (SIM) card, secure digital card (SD card), micro SD card, or solid-state drive (SSD), optical and magnetic media, others, or a combination thereof.

An input/output device is a mechanism or system for transferring data into or out of a computer. Exemplary input/output devices include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem. Input/output devices may be used to allow a user to control the instrument and receive data obtained from assays performed using the instruments and systems of the invention.

In certain aspects, the systems and instruments of the invention can include a fluidics module for interfacing with the cartridge in the cartridge holder. The fluidics module can, for example, introduce and manipulate samples and reagents in fluidic cartridges. The fluidics module can include one or more pipettors operably connected to reservoirs to dispense samples and reagents into and/or out of a fluidic cartridge.

In certain aspects, systems and instruments of the invention include one or more may conveyor or robotic arm. These arms can be used, for example, to seat and remove fluidic cartridges from the fluidic cartridge holder. The arms can be controlled using a computer system as described herein.

In certain aspects, the systems and instruments of the invention are modular, such that components can be introduced, removed, and replaced, for example on the rotating stages. For example, depending on the requirements of a particular assay, illumination zones and optical elements can be replaced, such that the system can provide optimal excitation light to a series of fluorescent dyes and detect emission light using the appropriate filters. Similarly, thermal elements can be added or removed to provide more complex thermal assays and/or perform amplification reactions on more than one fluidic cartridge simultaneously.

### EXAMPLE

### Example 1—Performing dPCR in PIP Encapsulated Droplets

A primer mix is prepared with forward and reverse primers. The forward (KRAS-G12F) and reverse primers (KRAS-G12R) are specific for sequences in a target nucleic acid encoding a KRAS gene, which is part of the RAS/MAPK signaling pathway. KRAS is known as an oncogene, i.e., a gene that when mutated has to potential to cause normal cells to become cancerous. The KRAS G12C mutation accounts for nearly half of all KRAS mutations in patients with non-small cell lung cancer. The primer mix includes 20 μL of each primer and water for a total volume of 100 μL.

An aqueous solution is then prepared in a sample tube with 25 μL of 1.2×Buffer 1, which includes template particles and dNTPs, 1.35 μL of the primer mix, 0.75 μL of a KRAS-G12 specific TaqMan fluorescent probe, 0.6 μL of FastStart Taq polymerase, and around 0.28 μL of fragmented genomic DNA from a patient. The aqueous solution is mixed 10 times using a pipette with P200 low retention tips.

150 μL of HFE7500 fluorinated oil is added to the aqueous solution.

The aqueous solution and fluorinated oil are vortexed for 2 minutes at 3000 rpm, during which the template particles cause the spontaneous formation of monodisperse droplets. The vortexed tube is placed upward to let the emulsion of droplets cream. 130 μL of the oil is removed from the tube.

The tube is then placed in a thermal cycler. The thermal cycler performs a FastTaq hot start at 95° C. for five minutes, followed by denaturing at 94° C. for 30 seconds, which is followed by annealing/extension at 60° C. for 1 minute. The denaturing and annealing/extension is iteratively performed 40 times.

The resulting signal from the hydrolyzed TaqMan probes is detected by imaging the droplets with a Luna Dual Fluorescence Cell Counter.

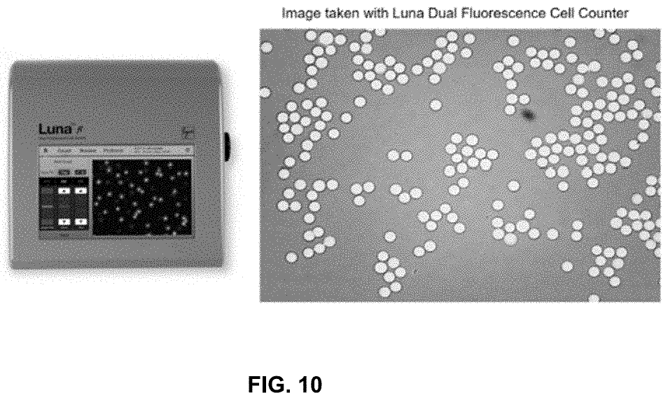

FIG. 10 shows the Luna Cell Counter and an image of the droplets obtained using it.

The resulting fluorescent signals are used to quantify the amount of KRAS target nucleic acid in a sample.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A method to detect a target nucleic acid, the method comprising:
preparing an aqueous solution comprising a target nucleic acid derived from a biological sample, PCR reagents, template particles, primers specific for the target nucleic acid, and fluorescent probes;
combining the aqueous solution with an oil in a vessel to create a mixture;
shearing the mixture to form a plurality of water-in-oil partitions, wherein each of the partitions includes a single target nucleic acid, PCR reagents, a template particle, primers specific for the target nucleic acid, and at least one fluorescent probe;
hybridizing the primers and fluorescent probes to the target nucleic acid;
amplifying the target nucleic acids in the partitions, thereby hydrolyzing the probes to release a fluorescent label;
identifying partitions with a fluorescent signal from the fluorescent label to detect the presence of the target nucleic acid in the sample.

2. The method of claim 1, wherein the water-in-oil partitions are formed simultaneously.

3. The method of claim 1, wherein the template particles template the formation of the droplets and segregate the microbial nucleic acid inside one of the droplets away from other nucleic acids present in the sample.

4. The method of claim 1, wherein the fluorescent signal is detected using a fluorometer.

5. The method of claim 4, wherein the fluorometer is a fluorescent cell counter.

6. The method of claim 1, further comprising quantifying the amount of target nucleic acid in the sample.

7. The method of claim 6, wherein quantifying the amount of target nucleic acid includes counting the number of droplets that produce a detectable signal and the number of droplets that do not produce a detectable signal.

8. The method of claim 7, wherein the target nucleic acid is loaded into the partitions at a limiting dilution.

9. The method of claim 1, wherein further comprising detecting the presence of two or more different target nucleic acids in a sample.

10. The method of claim 9, wherein shearing the mixture forms a plurality of partitions that each include one of the different target nucleic acids.

11. The method of claim 10, wherein the partitions include a plurality of hydrolysis probes, wherein each probe binds to a different target nucleic acid and includes a different fluorescent label.

12. The method of claim 11, wherein identifying the presence of the target nucleic acids in the sample includes imaging the partitions to detect the fluorescence emission of each different fluorescent label.

13. The method of claim 12, further comprising quantifying the amount of each different target nucleic acid in the sample.

14. A method to detect microbial nucleic acid, the method comprising:
obtaining a sample comprising a microbial nucleic acid;
partitioning the sample to form a plurality of droplets simultaneously, wherein the microbial nucleic acid is segregated inside one of the droplets;
binding, inside the droplet, the microbial nucleic acid with a capture probe;
amplifying bound microbial nucleic acid to create an amplicon; and
detecting the amplicon to thereby detect the microbial nucleic acid.

15. The method of claim 14, wherein the microbial nucleic acid comprises 16s rDNA.

16. The method of claim 14, wherein amplifying the bound microbial nucleic acid is performed with PCR in the presence of a fluorophore and wherein said fluorophore is incorporated into the amplicon during amplification.

17. The method of claim 16, wherein the fluorophore comprises an intercalating dye.

18. The method of claim 16, wherein detecting the amplicon is achieved by sensing a fluorescent signal from the fluorophore, wherein the fluorescent signal is indicative of the amplicon.

19. The method of claim 14, further comprising:
combining template particles with the sample in a first fluid;
adding a second fluid that is immiscible with the first fluid to create a mixture; and
vortexing the mixture, thereby partitioning the sample to form the plurality of droplets.

20. The method of claim 19, wherein the template particles template the formation of the droplets and segregate the microbial nucleic acid inside one of the droplets away from other nucleic acids present in the sample.

21-50. (canceled)
